(19) United States
(12) Patent Application Publication
(10) Pub. No.: US 2024/0300935 A1
(43) Pub. Date: Sep. 12, 2024

(54) Nitrogen Oxide Donors of Beraprost Derivative and the Pharmaceutical Compositions with the Use Thereof

(71) Applicant: GUANGZHOU KEMROCMED CO., LTD., Guangzhou (CN)

(72) Inventors: Xiameng Huang, Guangzhou (CN); Jingfan Pan, Guangzhou (CN); Xiaochuan Wu, Guangzhou (CN)

(21) Appl. No.: 18/668,422

(22) Filed: May 20, 2024

Related U.S. Application Data
(63) Continuation of application No. PCT/CN2023/072256, filed on Jan. 16, 2023.

(30) Foreign Application Priority Data
Nov. 18, 2021 (CN) 202111366683.9

(51) Int. Cl. C07D 413/12; A61K 31/343; A61K 31/4245; A61P 9/12; A61P 13/12; A61P 19/10; C07D 307/77

(52) U.S. Cl. CPC C07D 413/12; A61K 31/343; A61K 31/4245; A61P 9/12; A61P 13/12; A61P 19/10; C07D 307/77

(57) ABSTRACT

The present invention discloses a series of new compounds containing beraprost and nitric oxide donors, as shown in formula I below, relating to the series of compounds and their pharmaceutical compositions and uses. The invention provides a class of beraprost sodium and NO donor combination drugs, solves the short half-life of clearance problem of beraprost sodium , more daily administration, efficacy of saturation capping effect, and rapid catabolism half-life of NO. The new compound could reduce the dose and frequency, at the same time in-situ generated of NO could induce relaxation smooth muscle effect, through dual action, showcasing the synergistic effect, improving the effectiveness and safety of the drug.

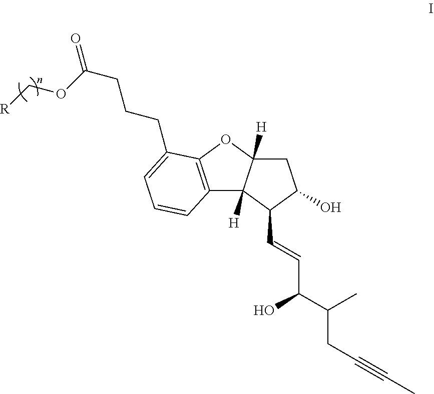

### CROSS-REFERENCE TO RELATED APPLICATIONS

The present application is a continuation application of PCT application No. PCT/CN2023/072256 filed on Jan. 16, 2023, which claims the benefit of Chinese Patent Application No. 202111366683.9 filed on Nov. 18, 2021. The contents of all of the aforementioned applications are incorporated by reference herein in their entirety.

### TECHNICAL FIELD

The present invention belongs to the field of biological medicine, and specifically relates to a nitrogen oxide donor beraprost derivative or its medicinal salts, pharmaceutical compositions and uses thereof.

### BACKGROUND TECHNOLOGY

Pulmonary hypertension (Pulmonary Hypertension, PH, including pulmonary hypertension Pulmonary Arterial Hypertension, PAH) is a type of diseases characterized by elevated pulmonary vascular resistance and right ventricular failure. The patient is confirmed to have a high short-survival mortality rate and is a malignant disease.

Currently clinically used in the treatment of pulmonary arterial hypertension are endothelin receptor antagonists (such as bosentan), phosphoesterase 5 inhibitors (such as Sildenafil), guanylate cyclase agonist (Leo), prostacyclin analogues (such as prostacyclin receptor agonist (sai), the mechanism of action of these drugs will eventually involve nitric oxide (Nitric oxide, NO) and cGMP pathway to relax endothelial blood vessels.

Among these drugs, prostaglandin analogue is the most effective and classic drugs, prostaglandin sodium (beraprost sodium) is the main application in clinical oral preparation, but due to its drug generation defects, requires multiple daily administration, therefore, researchers should conduct novel manipulations on the formulation of prostaglandin (such as sustained release Careload successfully marketed in Japan) and/or the structure (e.g. prostaglandin optical pure Esuberaprost failed in phase III clinical studies).

Because NO has the most critical role in the whole route of action, but because of its gas characteristics and difficult to administer, the donor (NO donor) mode is a new attempt to develop new drugs, such as the long-acting inhalant of liposome aerosols prepared by NO donors for the treatment of PAH (Nahar K, etc., Pharma Res.2016); The Latanoprost nitrate developed by Valeant Pharma is composed of Butylene glycol mononitrate, possessing a dual mechanism of action in the treatment of glaucoma: Latanoprost (latanoprost acid, Listed drugs) can act on the uveal scleral pathway, promoting the discharge of house water; Butanediol mononitrate (butanediol mononitrate) can release nitric oxide (NO), through the trabecular mesh and the xu lime tube (Schlemm's canal), Promote house water discharge. This two-pronged new approach has been demonstrated in clinical trials: latanoprost-NO prodrug provides better clinical advantages than single administration and was approved by the FDA in 2017 (trade name VYZULTA). Therefore, the modification of prostaglandins plus NO donor mode can increase the effectiveness of the drug through two pathways, which is a more convenient path for new drug development.

Beraprost, except for the treatment of pulmonary hypertension, it has also been used for malignant tumor metastasis (developed by United Therapeutics), atherosclerosis (developed by Kaken Pharma), hypertension (developed by Kaken Pharma and U nited Therap respectively), diabetic neuropathy (developed by Kaken Pharma), And nephritis and renal failure, Cerebrovascular dementia (CN 112691109A), Treatment of alcoholic fatty liver disease (HK1219665A) and other diseases. Meanwhile, NO donor drugs are also used to develop the treatment of various diseases such as anti-inflammatory and cardiovascular diseases (Megson IL & Webb DJ, Expert Opin Investig Drugs, 2002; Knox CD et al., MK 8150, J Am Heart Assoc, 2016). Therefore, both beta-prostanoid sodium and NO donors have the possibility to develop multiple therapeutic agents.

The invention is a series of nitric oxide for beraprost derivatives or its medicinal salt developed drugs, the series of compounds into the body into prostate and produce nitric oxide NO, can produce dual pharmacological effects, on the one hand can specifically combine with proprost receptor, play the role of vascular smooth muscle, on the other hand, these compounds in the body can release NO molecules, also play the role of endothelial cells cGMP way vasodastolic vessels, two mechanisms synergistic to achieve therapeutic effect.

These series of compounds can be used in the treatment of pulmonary hypertension, myocardial infarction, kidney disease, occlusive arteriosclerosis and other peripheral vascular diseases, and ophthalmic diseases (such as diabetic fundus lesions, glaucoma, etc.), osteoporosis, thrombotic vasculitis, thromboembolic diseases and other diseases of therapeutic drugs.

### SUMMARY

This application provides a new series of compounds of beraprost sodium combined with NO donor, such as the short half-life of clearance, the number of daily doses, the saturation capping effect, and the short half-life of rapid catabolismof gas in solution.

To achieve the above purpose, a nitric oxide donor type beraprost derivative or its medicinal salt as described in the following invention:

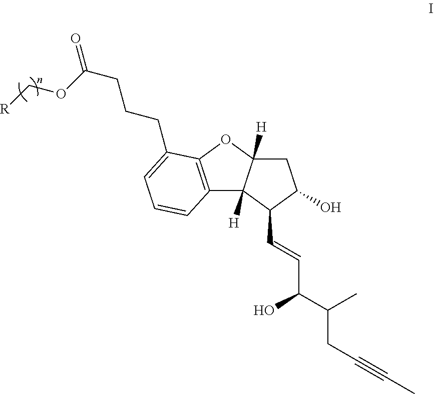

n is 0, 1, 2, 3 or 4;

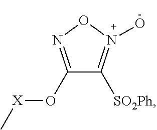

R is —X—ONO₂, —OC(O)—X—ONO₂, —O—X—ONO₂, or where X is a straight or branched chain of C₁C₁₀ alkyl, cycloalkyl, or —C₁-C₁₀ alkyl-aromatic ring-; where C₁-C₁₀ alkyl, C₅₋₇ cycloalkyl or aromatic ring may be substituted by one or more of the following substituents: the halogen atom, hydroxyl, carboxyl, cyanogen, or —(C₁-C₁₀ alkyl)—ONO₂.

The cycloalkyl group is preferably a C₅₋₇ Cycloalkyl, and the aromatic ring is the C₅₋₁₀ aromatic ring.

Further, the compound comprises any of the following specific structures:

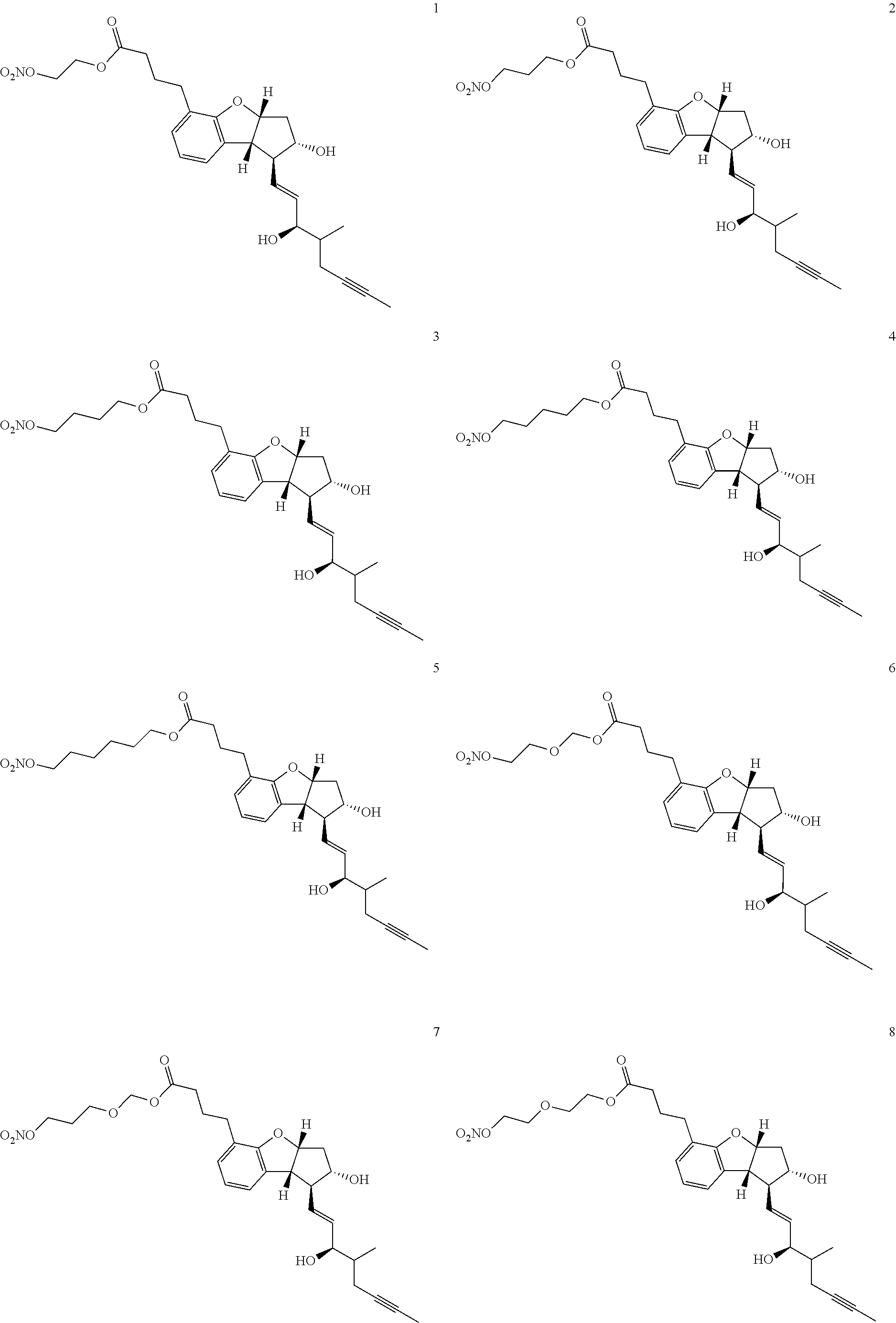

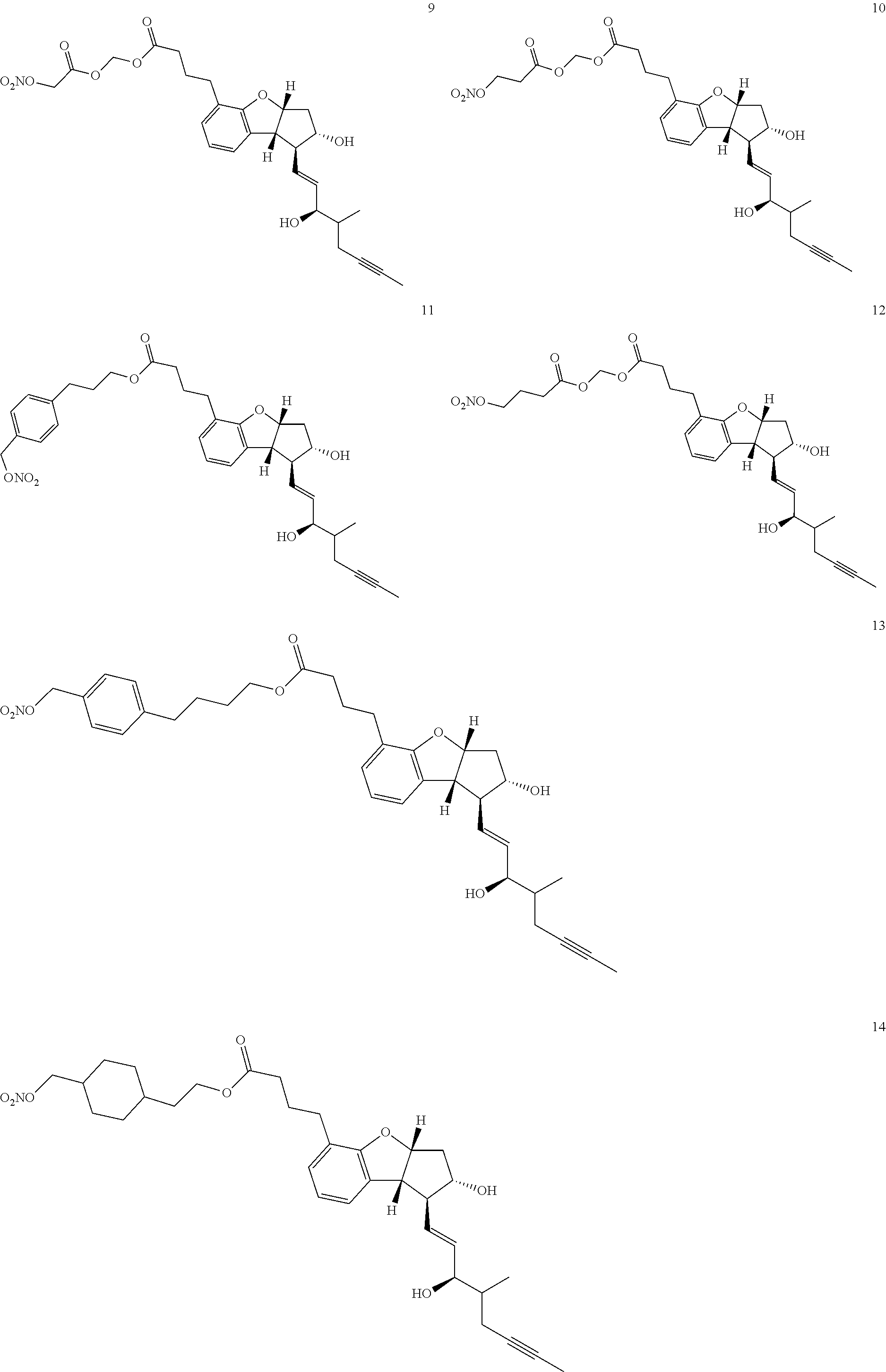

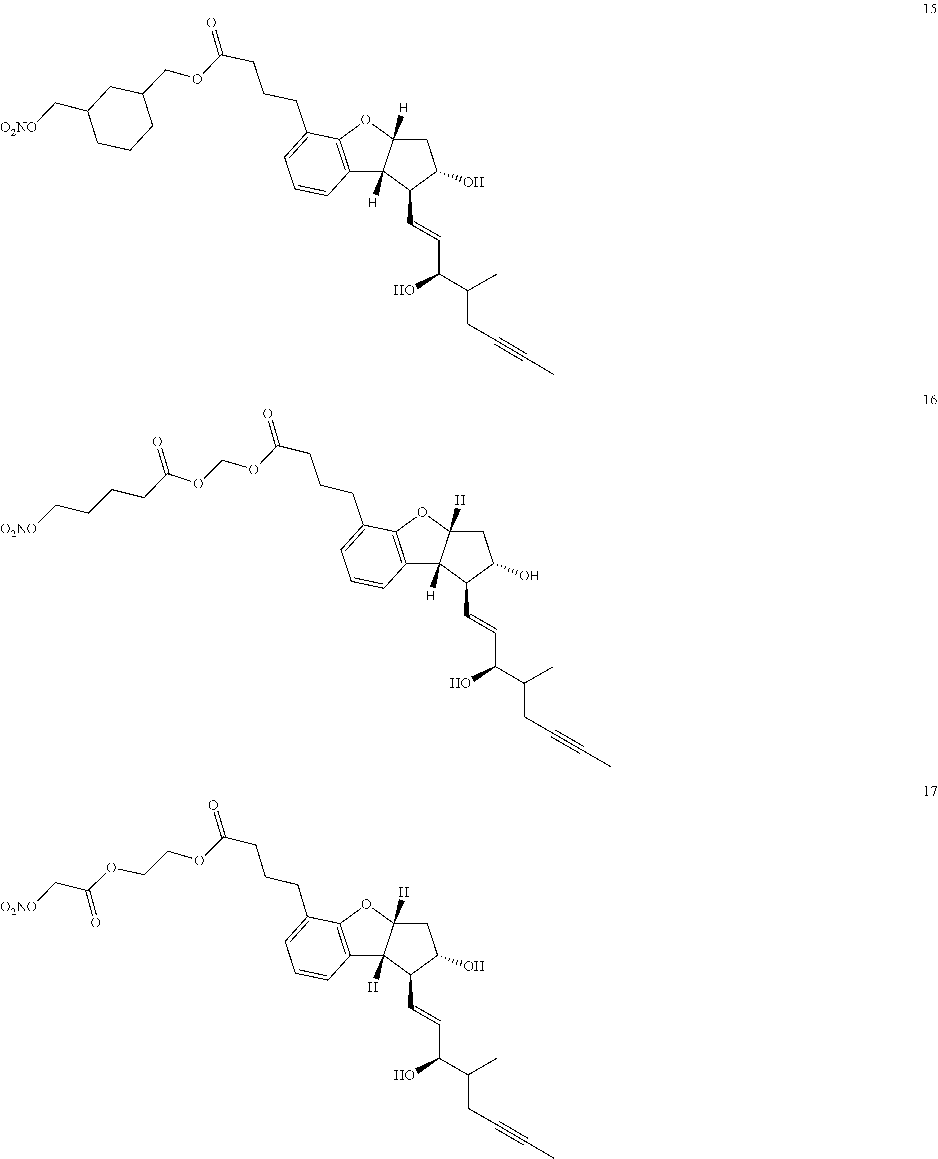

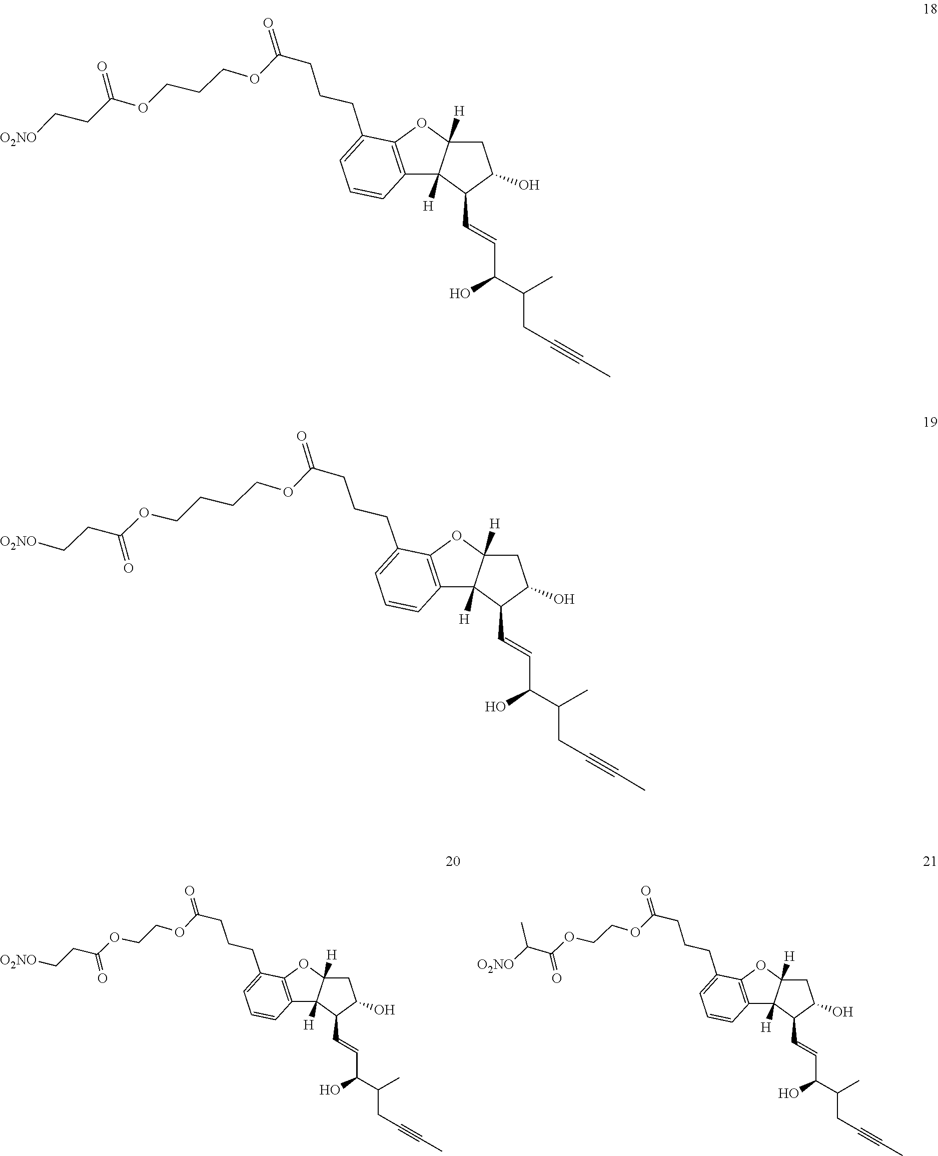

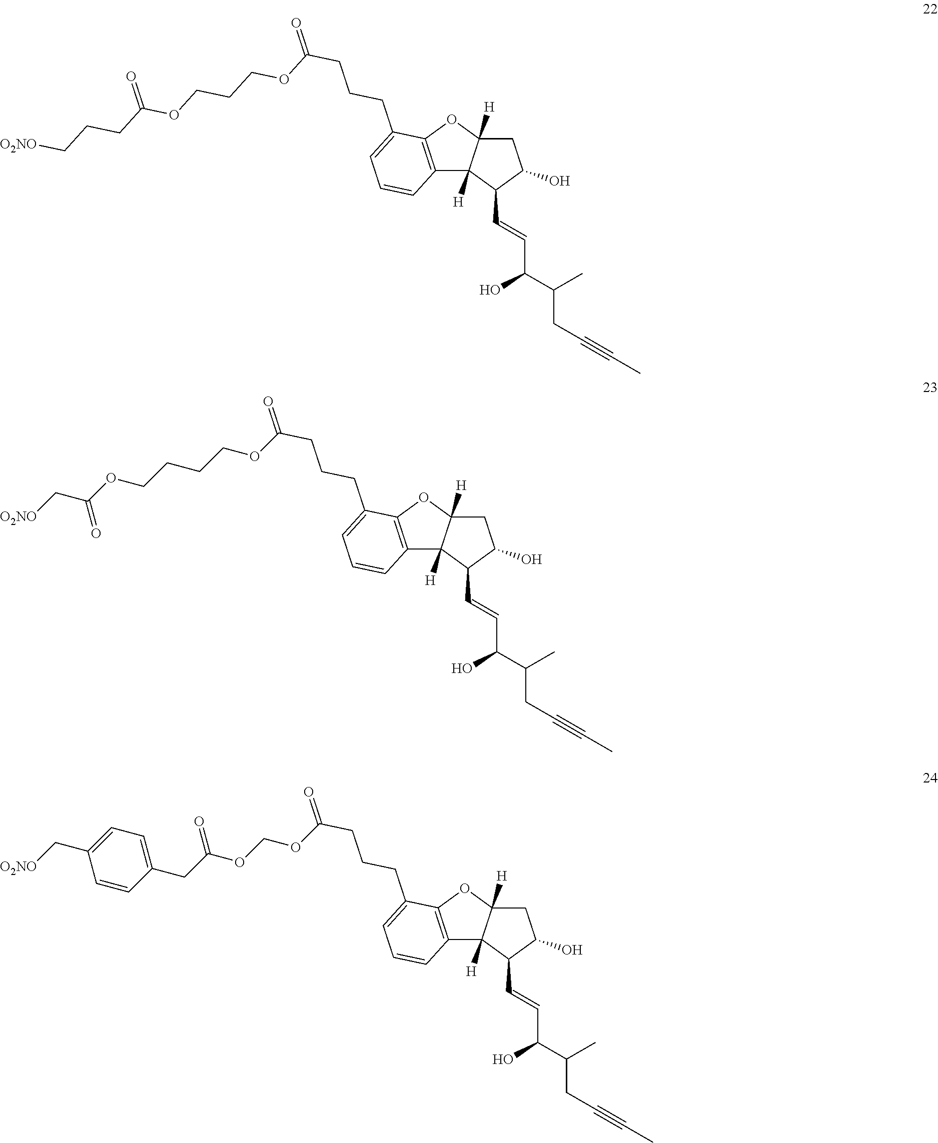

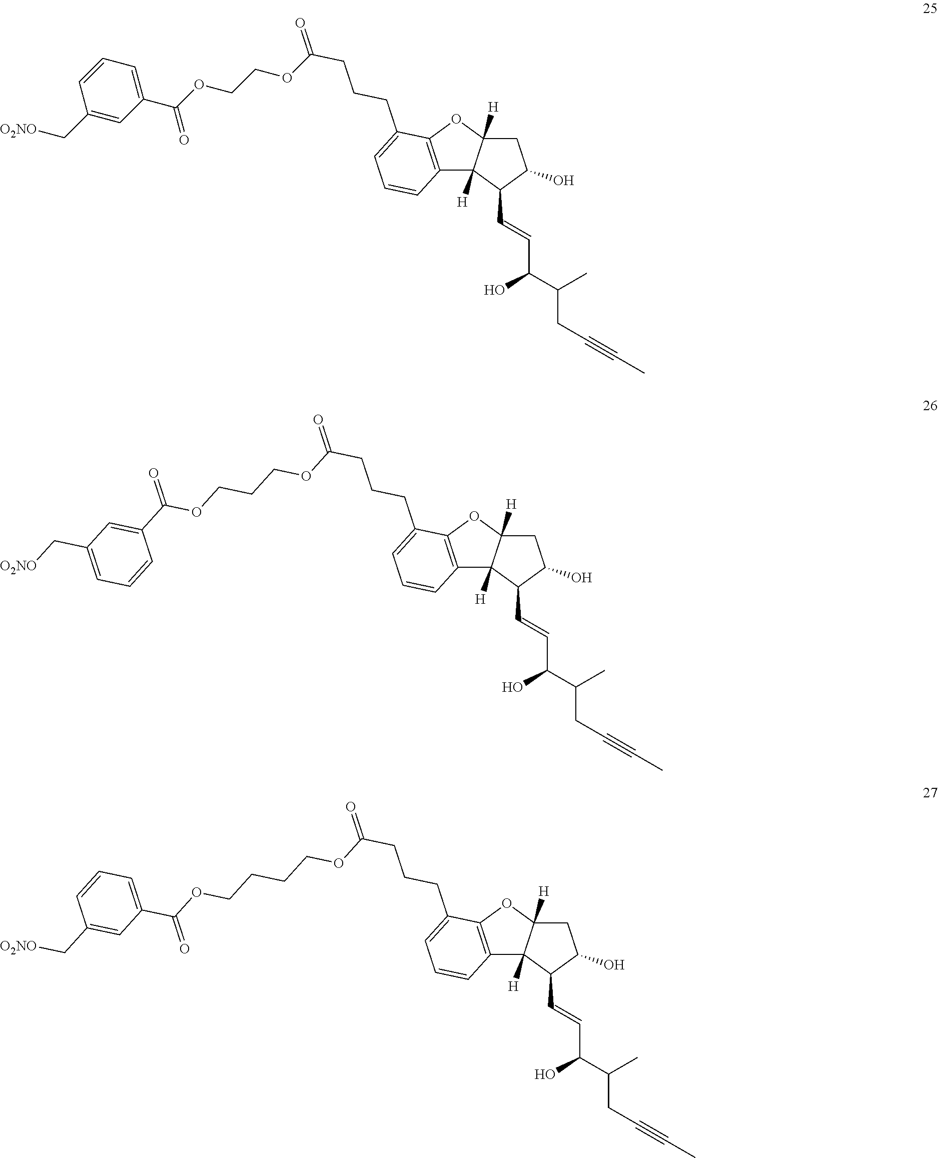

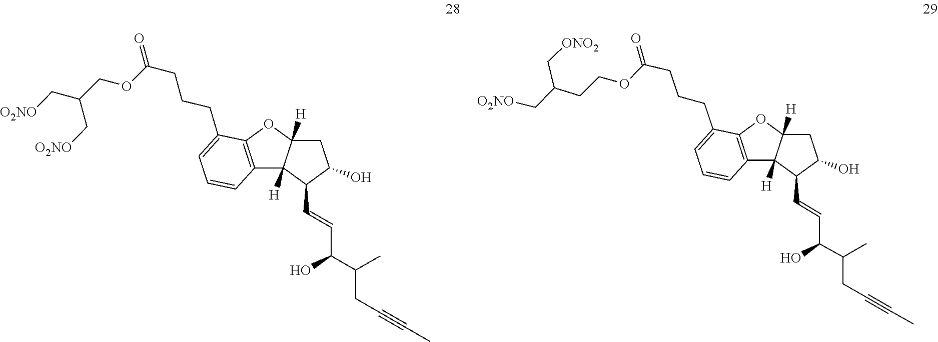

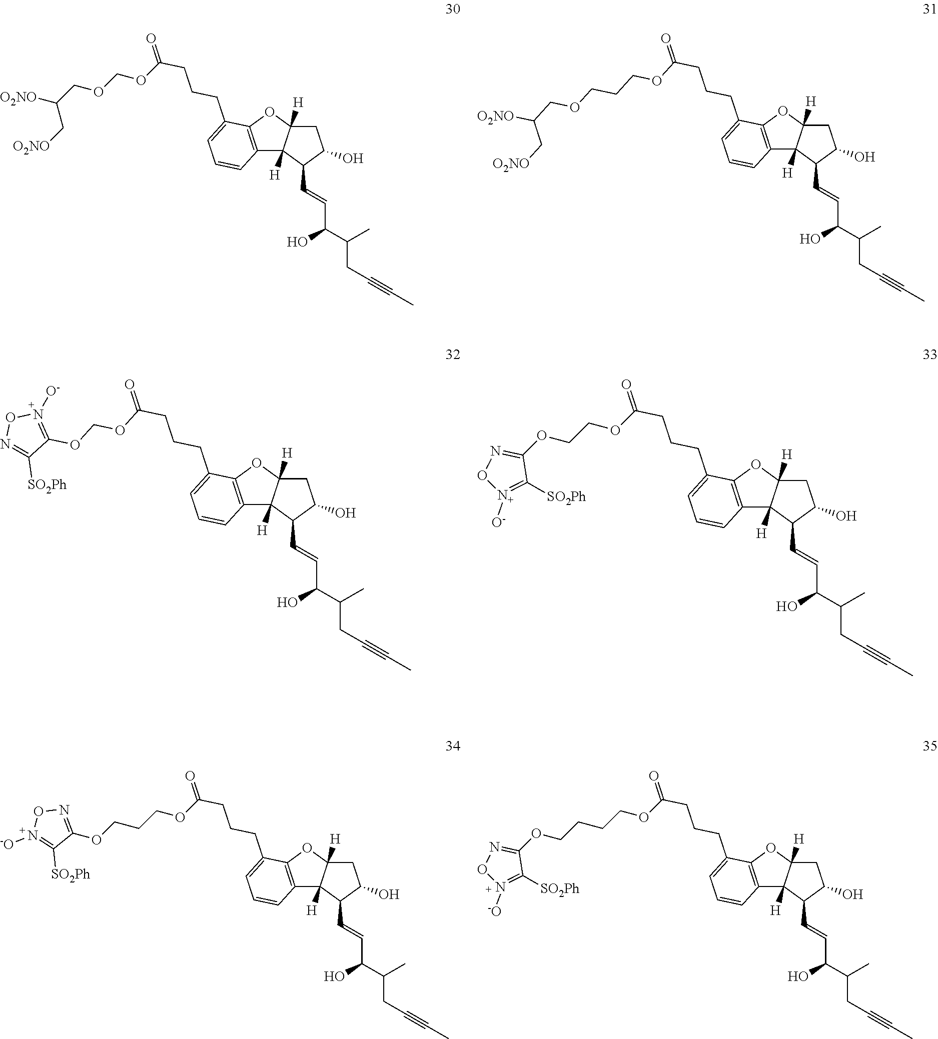

The pharmaceutically acceptable salt of the nitric oxide donor of beraprost derivatives referred to by the invention may be an acidic salt or an alkaline salt. Acid salts such as hydrochloric acid, hydrofluoric acid, hydrobromic acid, hydroiodized acid, sulfuric acid, pyrosulfuric acid, phosphoric acid or nitric acid, Or hydrogen bisulfate, Or acid addition salts formed with organic acids, such as formic acid, acetic acid, acetoacetic acid, pyrutronic acid, trifluoroacetic ic acid, propionic acid, butyric acid, prohexanoic acid, heptanic acid, hexanoic acid, lauric acid, benzoyl acid, salicylic acid, benzoic acid, cassia bark acid, cyclopane propanoic acid, 3-hydroxyl acid-2-naphthalic acid, nethoic acid, bisteric acid, 3-phenylalanic acid, terric acid, itacanteric acid, valecteric acid, 2,2,2, bisanteric acid, camphor Brain sulphonic acid, citric acid, tartaric acid, stearic acid, lactic acid, oxalic acid, malonic acid, succinic acid, malic acid, adipic acid, alginate, maleic acid, fumaric acid, D-gluconic acid, tonic acid, ascorbic acid, gluglytanic acid, glycerophosphoric acid, aspartic acid, sulfosalicylic acid, semisulfuric acid or thiocyanuric acid. Alkaline salts such as sodium ion, potassium ion, N-methylglucosine, dimethylglucosine, ethylgluconine, lysine, dicycloheximine, 1,6-adidiamine, ethanolamine, glucosamine, gluconine, sarcosine, serine, trihydroxymethylomethane, aminopropylene glycol, 1-amino-2,3,4-butantranol.

Another technical solution of the present application provides a pharmaceutical composition containing the above nitric oxide donor beraprost derivative, including a compound of the structure shown in formula I or its medicinable salt, and a medicinable carrier.

The carrier is a mixture of any one or more of a sustained release agent, excipients, filler, adhesive, wetting agent, disintegration agent, absorption promoter, adsorption carrier, surfactant and lubricant.

The drug composition, preferably either of the topical, oral, and injectable preparations.

The oral formulation is any of the granules, capsules, and tablets.

The pharmaceutical composition of the nitric oxide donor type beraprost derivative of the present invention, including its application as a pro-cyopxin analogue.

The drug composition of nitric oxide donor beraprost derivatives described in the present invention, including its application in the treatment of peripheral vascular diseases such as pulmonary artery, hypertension, myocardial infarction, renal disease, and ophthalmic diseases (such as diabetic fundus lesions, glaucoma, etc.), osteoporosis, thrombotic vasculitis, and thromboembolic diseases.

Beneficial effect: Compared with the prior art, the present invention has the following advantages:

The invention provides a class of beraprost sodium and NO donor combination drugs, overcomes the drawbacks of short clearance half-life of beraprost sodium, more daily administration, efficacy saturation capping effect, and rapid catabolized half-life of NO in solution. The new compound could reduce the dose and frequency, at the same time using the release of NO molecules to induce the relaxation smooth muscle effect through dual action, playing synergistic effect and improving the effectiveness and safety of the drug.

### BRIEF DESCRIPTION OF THE DRAWINGS

FIG. 1 shows the effect of in vivo murine hypoxic pulmonary hypertension;

FIG. 2 shows the mouse BMD data after treatment with compound 15;

FIG. 3 represents the effect of compound 15 on renal tubular epithelial cell proliferation in acute renal failure.

### DETAILED DESCRIPTION OF THE EMBODIMENTS

The invention is further explained in combination with embodiments.

### Example 1

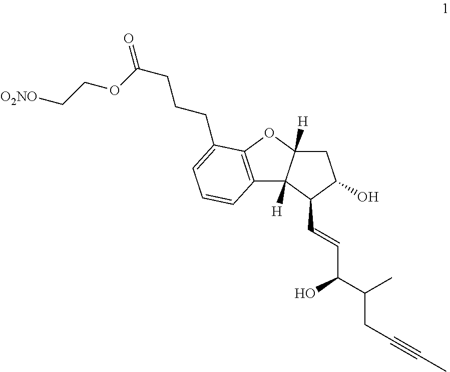

synthetic route:

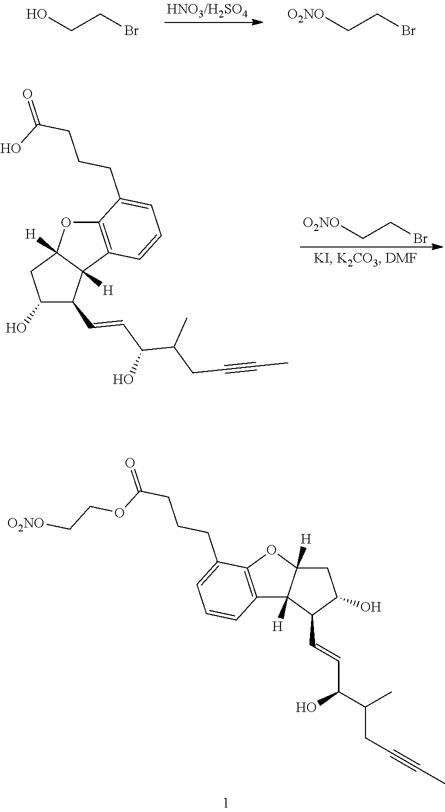

### Synthesis of Example 1

The concentrated sulfuric acid (13 mmol) was dissolved in dichloromethane, and the smoke nitric acid (14 mmol) was then slowly in at 0° C. After the reaction for 20 min, 2-bromoethanol (6 mmol) was added to the reaction solution. When the reaction was continued for 4 hours at 0° C., the reaction solution was slowly poured into ice water, extracted twice with dichloromethane (50 mL), and the organic phase was collected, washed once, saturated salt once, and dried to obtain product 2-bromoethyl nitrate.

beraprost (60 mg), dissolved in 2 mL anhydrous DMF, potassium iodide (75 m g), potassium carbonate (62 mg) and 2-bromoethyl nitrate (80 mg) dropped, moved to 50° C. stirring reaction for 2 h, TLC detection reaction complete, dry solvent, H PLC purified to Example 1.67% yield. ¹H NMR (300 MHz, DMSO-d) δ 7.38-7.22 (m, 2H), 7.00 (d, J=5.8 Hz, 1H), 5.85 (d, J=9.5 Hz, 2H), 4.63-4.51 (m, 2H), 4.38 (d, J=12.5 Hz, 1H), 4.23-4.15 (m, 2H), 3.49-3.36 (m, 2H), 2.95-2.76 (m, 2H), 2.61 (dq, J=12.5, 2.0 Hz, 1H), 2.56-2.39 (m, 4H), 2.28 (d, J=13.0 Hz, 1H), 2.01-1.99 (m, 1H), 1.95-1.76 (m, 5H), 1.03 (d, J=5.5 Hz, 3H). ESI-MS m/z: 510.2 [M+Na]⁺.

### Example 2

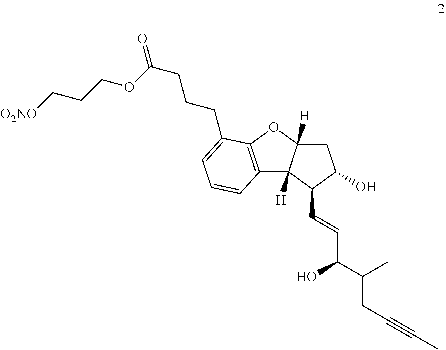

Referring to the synthesis method of Example 1, Example 2 can be prepared. ¹H NMR (300 MHz, DMSO-d) δ 7.35-7.24 (m, 2H), 7.04 (ddd, J=3.7, 2.7, 1.4 Hz, 1H), 5.88 (s, 1H), 5.82 (s, 1H), 4.71-4.52 (m, 3H), 4.35-4.10 (m, 6H), 2.86 (qd, J=12.3, 0.9 Hz, 2H), 2.61 (dq, J=12.5, 2.0 Hz, 1H), 2.51-2.37 (m, 4H), 2.36-2.15 (m, 3H), 1.94-1.74 (m, 6H), 1.00 (d, J=5.7 Hz, 3H).ESI-MS m/z: 524.2 [M+N a]⁺.

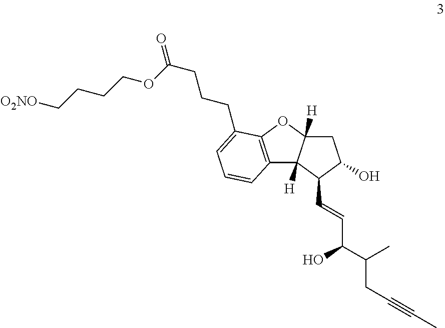

### Example 3

Example 3 may be prepared with reference to the synthesis method of Example 1. ¹H NMR (300 MHz, Methanol-d₄) δ 7.39-7.22 (m, 2H), 7.04 (d, J=3.1 Hz, 1H), 5.69-5.46 (m, 2H), 4.69-4.52 (m, 3H), 4.31-4.09 (m, 5H), 4.01 (d, J=12.5 Hz, 1H), 2.86 (qd, J=12.3, 0.9 Hz, 2H), 2.70 (dq, J=12.5, 2.0 Hz, 1H), 2.55 (dq, J=12.3, 1.9 Hz, 1H), 2.49-2.38 (m, 3H), 2.28 (d, J=13.0 Hz, 1H), 2.09-1.78 (m, 10H), 1.02 (d, J=5.3 Hz, 3H).

### Example 4

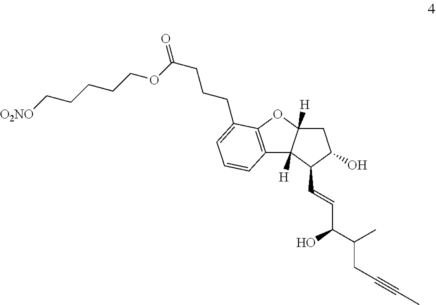

Referring to the synthesis method of Example 1, Example 4 may be prepared. ¹H NMR (300 MHZ, DMSO-d₄) δ 7.41-7.24 (m, 1H), 7.07 (d, J=7.4 Hz, 1H), 5.85 (d, J=7.5 Hz, 1H), 4.71-4.51 (m, 2H), 4.28-4.00 (m, 3H), 2.94-2.77 (m, 1H), 2.66 (dq, J=12.5, 2.0 Hz, 1H), 2.56-2.37 (m, 2H), 2.28 (d, J=13.0 Hz, 1H), 1.94-1.78 (m, 5H), 1.68-1.54 (m, 1H), 1.02 (d, J=5.5 Hz, 2H).

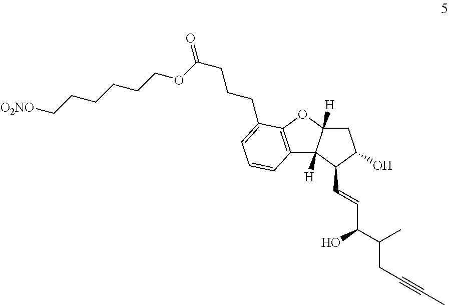

### Example 5

Referring to the synthesis method of Example 1, Example 5 may be prepared. ¹H NMR (300 MHz, DMSO-d₄) δ 7.37 (t, J=7.5 Hz, 1H), 7.24 (dd, J=7.5, 2.0 Hz, 1H), 7.02 (d, J=7.7 Hz, 1H), 5.65-5.42 (m, 2H), 4.71-4.52 (m, 3H), 4.32-4.02 (m, 6H), 2.93-2.77 (m, 2H), 2.66 (dq, J=12.4, 2.0 Hz, 1H), 2.52-2.26 (m, 5H), 1.95-1.81 (m, 5H), 1.68-1.59 (m, 4H), 1.53-1.35 (m, 4H), 1.00 (d, J=6.5 Hz, 3H).

### Example 6

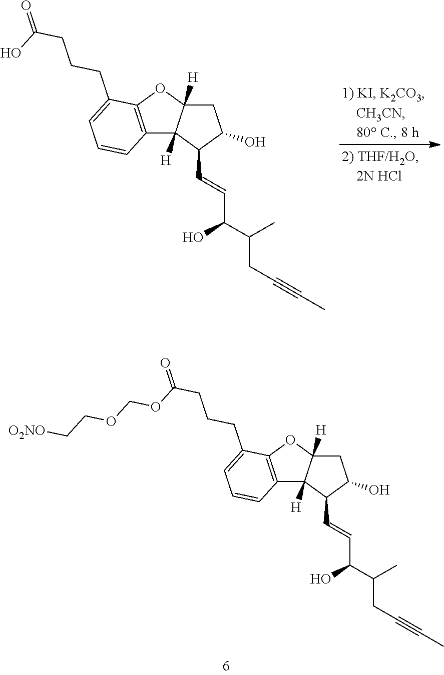

### Synthesis of Example 6

beraprost (60 mg), dissolved in 2 mL anhydrous acetonitrile, added potassium iodide (75 m g), potassium carbonate (62 mg), stirred at room temperature for 10 min, add 2-chloromethyl ethyl nitrate (15 mg), placed at 60° C. reaction for 8 h, stopped, dried solvent, methyl chloride, washed twice, saturated salt washed once, organic phase concentrated, H PLC purified to Example 6.42% yield. ¹H NMR (300 MHz, DMSO-d) δ 7.11-7.01 (m, 2H), 6.95 (dq, J=7.7, 1.2 Hz, 1H), 5.90-5.68 (m, 2H), 5.09 (s, 2H), 4.66-4.56 (m, 2H), 4.22-4.09 (m, 2H), 3.87 (t, J=6.2 Hz, 2H), 2.88-2.68 (m, 3H), 2.46 (t, J=7.1 Hz, 2H), 2.21 (dp, J=6.2, 2.0 Hz, 2H), 2.11 (t, J=4.7 Hz, 2H), 2.05-1.85 (m, 3H), 1.65 (t, J=2.0 Hz, 3H), 1.02 (d, J=6.8 Hz, 3H).

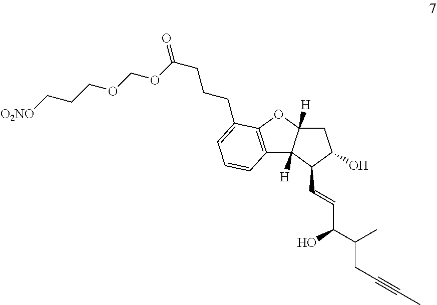

### Example 7

Referring to the synthesis method of Example 6, Example 7 can be prepared. ¹H NMR (300 MHZ, DMSO-d) δ 7.28-7.08 (m, 2H), 7.05 (dq, J=7.7, 1.2 Hz, 1H), 5.89-5.58 (m, 2H), 5.06 (q, J=2.7 Hz, 2H), 4.94 (q, J=4.5 Hz, 1H), 4.37 (t, J=6.1 Hz, 2H), 4.22-4.04 (m, 2H), 3.59 (t, J=6.1 Hz, 2H), 3.43 (dd, J=5.5, 4.2 Hz, 1H), 2.92-2.66 (m, 3H), 2.46 (t, J=7.1 Hz, 2H), 2.27-2.16 (m, 2H), 2.16-1.85 (m, 7H), 1.65 (t, J=2.0 Hz, 3H), 1.01 (d, J=6.5 Hz, 3H).

### Example 8

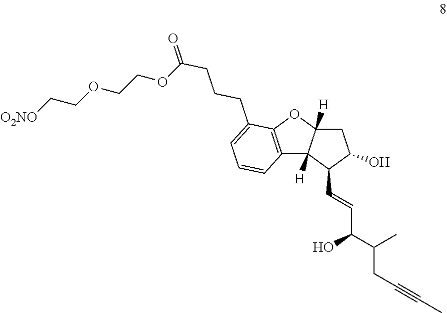

Referring to the synthesis method according to Example 6, the compound 8 can be produced. ESI-MS m/z: 513.3 [M+H]⁺. ¹H NMR (300 MHz, DMSO-d) δ 7.25-7.04 (m, 2H), 7.00 (dq, J=7.7, 1.0 Hz, 1H), 5.91-5.66 (m, 2H), 4.94 (q, J=4.5 Hz, 1H), 4.57 (t, J=6.2 Hz, 2H), 4.30-4.04 (m, 4H), 3.82 (t, J=6.2 Hz, 2H), 3.72 (t, J=6.2 Hz, 2H), 3.43 (dd, J=5.5, 4.2 Hz, 1H), 2.86-2.68 (m, 3H), 2.41 (t, J=7.0 Hz, 2H), 2.21 (dp, J=6.2, 2.0 Hz, 2H), 2.18-2.07 (m, 2H), 2.07-1.85 (m, 3H), 1.66 (t, J=2.0 Hz, 3H), 1.01 (d, J=6.9 Hz, 3H).

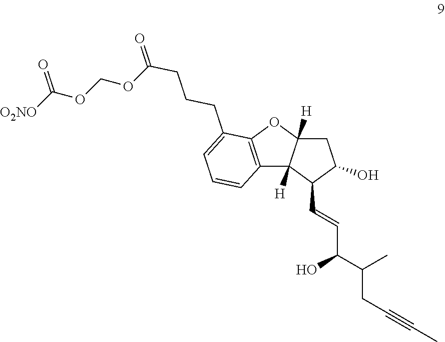

### Example 9

Referring to the synthesis method of Example 6, Example 9 can be prepared. ¹H NMR (300 MHZ, DMSO-d) δ 7.16-7.02 (m, 2H), 6.95 (dq, J=7.7, 1.2 Hz, 1H), 5.98-5.66 (m, 4H), 5.11-4.87 (m, 3H), 4.24-4.03 (m, 2H), 3.43 (dd, J=5.5, 4.2 Hz, 1H), 2.77 (dqd, J=30.2, 6.5, 6.1, 1.1 Hz, 3H), 2.46 (t, J=7.1 Hz, 2H), 2.21 (dp, J=6.2, 2.0 Hz, 2H), 2.11 (t, J=4.7 Hz, 2H), 2.02-1.87 (m, 3H), 1.55 (t, J=2.0 Hz, 3H), 1.02 (d, J=6.8 Hz, 3H).

### Example 10

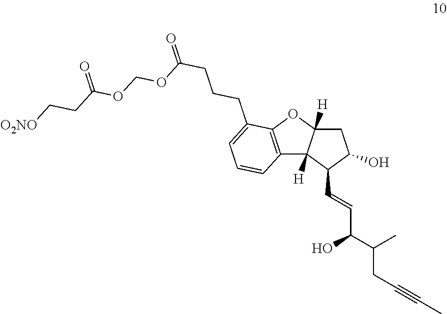

Referring to the synthesis method of Example 6, Example 10 can be prepared. ¹H NMR (300 MHz, Chloroform-d) δ 7.16-7.02 (m, 2H), 6.95 (ddt, J=6.0, 2.7, 0.9 Hz, 1H), 5.93-5.61 (m, 4H), 4.76-4.42 (m, 3H), 4.29-4.04 (m, 2H), 3.92 (d, J=5.5 Hz, 1H), 3.43 (dd, J=5.5, 4.2 Hz, 1H), 2.96-2.63 (m, 5H), 2.45 (td, J=7.0, 0.9 Hz, 2H), 2.30-2.06 (m, 4H), 2.06-1.84 (m, 3H), 1.57 (t, J=2.0 Hz, 3H), 1.00 (d, J=6.2 Hz, 3H).

### Example 11

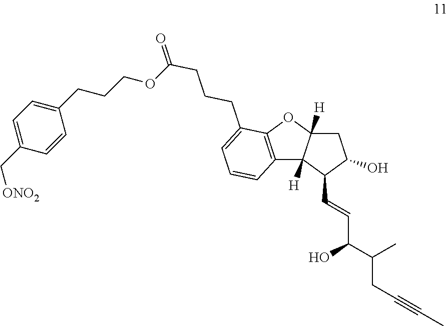

Referring to the synthesis method of Example 1, embodiment 11 may be prepared. ¹H NMR (300 MHz, DMSO-d) δ 7.49-7.30 (m, 2H), 7.18-7.02 (m, 4H), 6.97 (ddt, J=7.0, 1.9, 1.0 Hz, 1H), 5.78 (qd, J=15.6, 6.2 Hz, 2H), 5.52-5.24 (m, 2H), 4.80 (d, J=6.2 Hz, 1H), 4.39-4.02 (m, 5H), 3.42 (dd, J=5.5, 4.2 Hz, 1H), 2.89-2.56 (m, 5H), 2.40 (t, J=7.1 Hz, 2H), 2.26-1.85 (m, 9H), 1.61 (t, J=2.0 Hz, 3H), 1.01 (d, J=6.7 Hz, 3H).

### Example 12

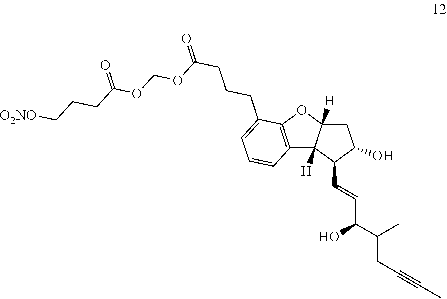

Example 12 can be prepared with reference to the synthesis method of Example 1. ¹H NMR (300 MHz, DMSO-d) δ 7.13-7.02 (m, 2H), 6.95 (ddt, J=5.5, 3.3, 0.9 Hz, 1H), 5.89-5.57 (m, 4H), 4.94 (dt, J=5.1, 4.3 Hz, 1H), 4.42 (qt, J=10.4, 6.1 Hz, 2H), 4.24-4.06 (m, 2H), 3.43 (dd, J=5.5, 4.2 Hz, 1H), 2.94-2.63 (m, 3H), 2.63-2.39 (m, 4H), 2.26-1.79 (m, 9H), 1.62 (t, J=2.0 Hz, 3H), 0.99 (d, J=6.8 Hz, 3H).

### Example 13

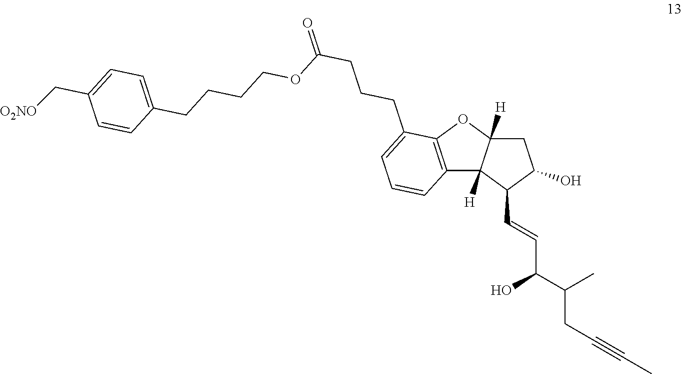

Referring to the synthesis method to the embodiment 13 can be prepared. ¹H NMR (500 MHZ, Chloroform-d) δ 7.51-7.30 (m, 2H), 7.19-7.05 (m, 4H), 6.97 (ddt, J=7.0, 2.0, 1.0 Hz, 1H), 5.78 (qd, J=15.6, 6.2 Hz, 2H), 5.52-5.31 (m, 2H), 4.94 (dt, J=5.1, 4.2 Hz, 1H), 4.22-4.05 (m, 5H), 3.42 (dd, J=5.5, 4.2 Hz, 1H), 2.87-2.68 (m, 3H), 2.62 (tq, J=6.5, 1.0 Hz, 2H), 2.40 (t, J=7.1 Hz, 2H), 2.21 (dp, J=5.9, 2.0 Hz, 2H), 2.16-2.01 (m, 2H), 2.01-1.87 (m, 3H), 1.81-1.60 (m, 7H), 1.02 (d, J=6.7 Hz, 3H).

### Example 14

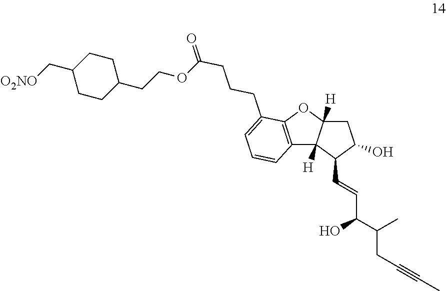

Referring to the synthesis method of Example 1, the compound 14 can be produced. ¹H NMR (300 MHz, DMSO-d) δ 7.22-6.99 (m, 2H), 6.97 (ddt, J=7.3, 1.8, 0.9 Hz, 1H), 5.78 (qd, J=15.6, 6.2 Hz, 2H), 4.94 (dt, J=5.0, 4.2 Hz, 1H), 4.29-4.15 (m, 3H), 4.15-4.03 (m, 3H), 3.42 (dd, J=5.5, 4.2 Hz, 1H), 2.89-2.66 (m, 3H), 2.40 (t, J=7.0 Hz, 2H), 2.31-2.18 (m, 2H), 2.18-1.84 (m, 6H), 1.76-1.33 (m, 14H), 1.04 (d, J=6.1 Hz, 3H).

### Example 15

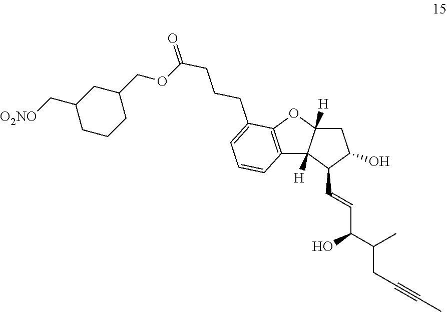

Referring to the synthesis method of Example 1, the compound 15 can be produced. ¹H NMR (300 MHz, DMSO-d) δ 7.21-7.05 (m, 2H), 6.97 (ddt, J=5.6, 3.5, 1.1 Hz, 1H), 5.78 (qd, J=15.6, 6.2 Hz, 2H), 4.94 (dt, J=5.0, 4.2 Hz, 1H), 4.34-4.10 (m, 5H), 3.75 (dd, J=10.5, 6.3 Hz, 1H), 3.42 (dd, J=5.5, 4.2 Hz, 1H), 2.89-2.66 (m, 3H), 2.41 (t, J=7.1 Hz, 2H), 2.21 (dp, J=6.2, 2.0 Hz, 2H), 2.17-1.82 (m, 7H), 1.82-1.59 (m, 5H), 1.59-1.39 (m, 6H), 1.01 (d, J=6.7 Hz, 3H).

### Example 16

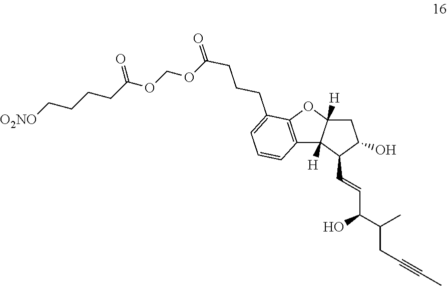

Referring to the synthesis method of Example 6, the compound 16 can be produced. ¹H NMR (300 MHz, DMSO-d) δ 7.13-7.04 (m, 2H), 6.97 (ddt, J=5.7, 3.5, 1.1 Hz, 1H), 5.87-5.62 (m, 4H), 4.94 (dt, J=5.1, 4.3 Hz, 1H), 4.32 (t, J=6.0 Hz, 2H), 4.23-4.05 (m, 2H), 3.92 (d, J=5.5 Hz, 1H), 2.91-2.66 (m, 3H), 2.51-2.32 (m, 4H), 2.26-2.17 (m, 2H), 2.17-1.75 (m, 9H), 1.62 (t, J=2.0 Hz, 3H), 0.99 (d, J=6.1 Hz, 3H).

### Example 17

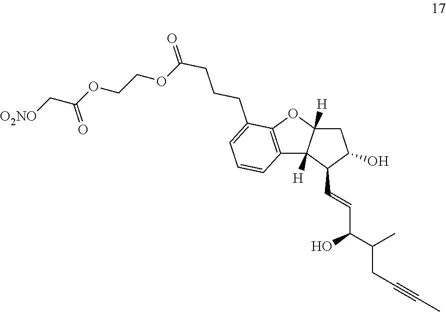

Referring to the synthesis method of Example 6, the compound 17 can be produced. ¹H NMR (300 MHz, DMSO-d) δ 7.13-7.00 (m, 2H), 6.96 (ddt, J=6.2, 2.9, 1.1 Hz, 1H), 5.89-5.63 (m, 2H), 5.04-4.83 (m, 3H), 4.69 (d, J=6.2 Hz, 1H), 4.48-4.28 (m, 4H), 4.22-4.03 (m, 2H), 3.92 (d, J=5.5 Hz, 1H), 2.93-2.60 (m, 3H), 2.40 (t, J=7.1 Hz, 2H), 2.25-2.07 (m, 4H), 2.07-1.86 (m, 3H), 1.57 (t, J=2.0 Hz, 3H), 1.00 (d, J=6.2 Hz, 3H).

### Example 18

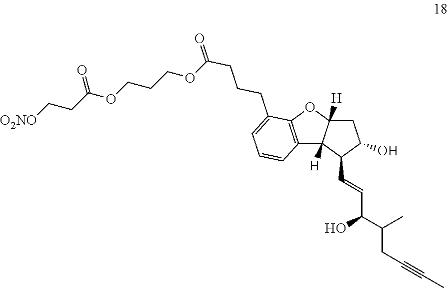

Referring to the synthesis method of Example 6, the compound 18 can be produced. ¹H NMR (300 MHz, DMSO-d) δ 7.18-7.02 (m, 2H), 6.97 (ddt, J=5.6, 3.5, 1.1 Hz, 1H), 5.78 (qd, J=15.6, 6.2 Hz, 2H), 4.94 (dt, J=5.0, 4.2 Hz, 1H), 4.71-4.52 (m, 2H), 4.23-4.04 (m, 6H), 3.43 (dd, J=5.5, 4.2 Hz, 1H), 2.92-2.66 (m, 5H), 2.40 (t, J=7.1 Hz, 2H), 2.21 (dp, J=6.2, 2.0 Hz, 2H), 2.18-1.85 (m, 7H), 1.60 (t, J=2.0 Hz, 3H), 1.04 (d, J=6.8 Hz, 3H).

### Example 19

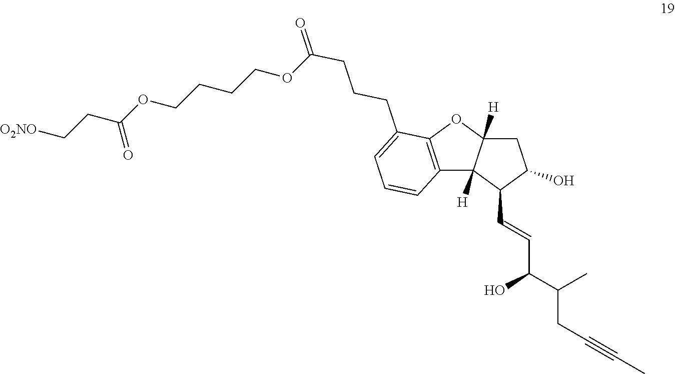

Referring to the synthesis method of Example 6, the compound 19 can be produced. ¹H NMR (300 MHz, DMSO-d) δ 7.15-7.02 (m, 2H), 6.97 (ddt, J=7.0, 1.9, 1.0 Hz, 1H), 5.78 (qd, J=15.6, 6.2 Hz, 2H), 4.94 (dt, J=5.1, 4.2 Hz, 1H), 4.61 (t, J=7.1 Hz, 2H), 4.26-4.04 (m, 6H), 3.42 (dd, J=5.5, 4.2 Hz, 1H), 2.89-2.67 (m, 5H), 2.39 (t, J=7.0 Hz, 2H), 2.21 (dp, J=6.2, 2.0 Hz, 2H), 2.17-1.87 (m, 5H), 1.87-1.73 (m, 4H), 1.60 (t, J=2.0 Hz, 3H), 0.98 (d, J=6.7 Hz, 3H).

### Example 20

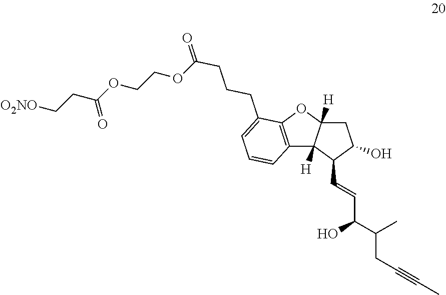

Referring to the synthesis method of Example 6, the compound 20 can be produced. ¹H NMR (300 MHz, DMSO-d) δ 7.13-7.02 (m, 2H), 6.96 (ddt, J=5.5, 3.3, 1.0 Hz, 1H), 5.78 (qd, J=15.6, 6.2 Hz, 2H), 4.94 (dt, J=5.0, 4.2 Hz, 1H), 4.71-4.51 (m, 2H), 4.40-4.26 (m, 4H), 4.26-4.05 (m, 2H), 3.43 (dd, J=5.5, 4.2 Hz, 1H), 2.91-2.63 (m, 5H), 2.40 (t, J=7.1 Hz, 2H), 2.21 (dp, J=6.2, 2.0 Hz, 2H), 2.20-1.85 (m, 5H), 1.65 (t, J=2.0 Hz, 3H), 1.02 (d, J=6.7 Hz, 3H).

### Example 21

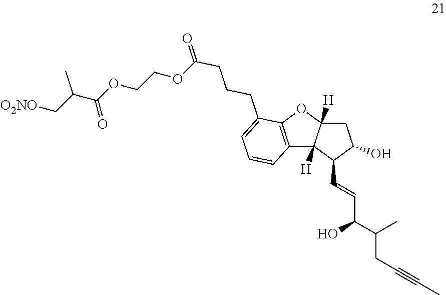

Referring to the synthesis method of Example 6, the compound 21 can be produced. ¹H NMR (300 MHz, DMSO-d) δ 7.20-7.10 (m, 2H), 7.01 (ddt, J=5.5, 3.3, 1.0 Hz, 1H), 5.78 (qd, J=15.6, 6.2 Hz, 2H), 5.25 (q, J=6.6 Hz, 1H), 4.94 (dt, J=5.1, 4.3 Hz, 1H), 4.52-4.41 (m, 2H), 4.41-4.26 (m, 2H), 4.26-4.05 (m, 2H), 3.43 (dd, J=5.5, 4.2 Hz, 1H), 2.91-2.67 (m, 3H), 2.40 (t, J=7.0 Hz, 2H), 2.21 (dp, J=6.2, 2.0 Hz, 2H), 2.17-1.84 (m, 5H), 1.65 (t, J=2.0 Hz, 3H), 1.47 (d, J=6.6 Hz, 3H), 1.00 (d, J=6.2 Hz, 3H).

### Example 22

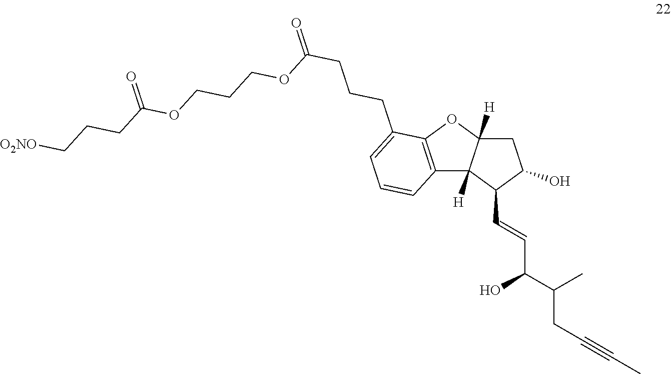

Referring to the synthesis method of Example 6, the compound 22 can be produced. ¹H NMR (300 MHz, DMSO-d) δ 7.21-7.08 (m, 2H), 7.00 (ddt, J=6.9, 1.9, 1.0 Hz, 1H), 5.78 (qd, J=15.6, 6.2 Hz, 2H), 4.94 (dt, J=5.0, 4.2 Hz, 1H), 4.54-4.31 (m, 2H), 4.22-4.03 (m, 6H), 3.42 (dd, J=5.5, 4.2 Hz, 1H), 2.91-2.62 (m, 3H), 2.49 (t, J=7.0 Hz, 2H), 2.40 (t, J=7.1 Hz, 2H), 2.21 (dp, J=6.2, 2.0 Hz, 2H), 2.18-1.82 (m, 9H), 1.55 (t, J=2.0 Hz, 3H), 1.00 (d, J=6.2 Hz, 3H).

### Example 23

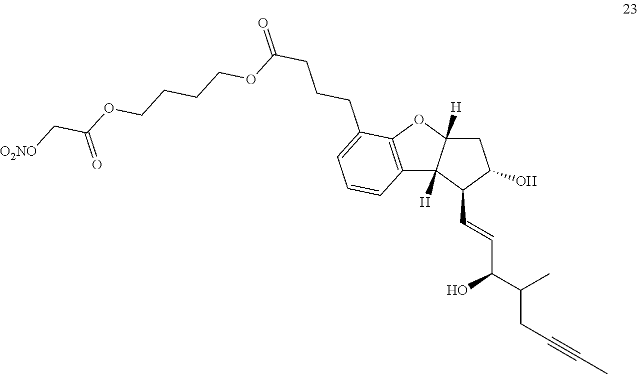

Referring to the synthesis method of Example 6, the compound 23 can be produced. ¹H NMR (300 MHz, DMSO-d) δ 7.17-7.08 (m, 2H), 7.00 (ddt, J=5.6, 3.5, 1.1 Hz, 1H), 5.78 (qd, J=15.6, 6.2 Hz, 2H), 5.09-4.87 (m, 3H), 4.27-4.05 (m, 6H), 3.43 (dd, J=5.5, 4.2 Hz, 1H), 2.86 -2.67 (m, 3H), 2.38 (t, J=7.1 Hz, 2H), 2.21 (dp, J=6.2, 2.0 Hz, 2H), 2.17-1.87 (m, 5H), 1.87-1.72 (m, 4H), 1.61 (t, J=2.0 Hz, 3H), 1.01 (d, J=6.7 Hz, 3H).

### Example 24

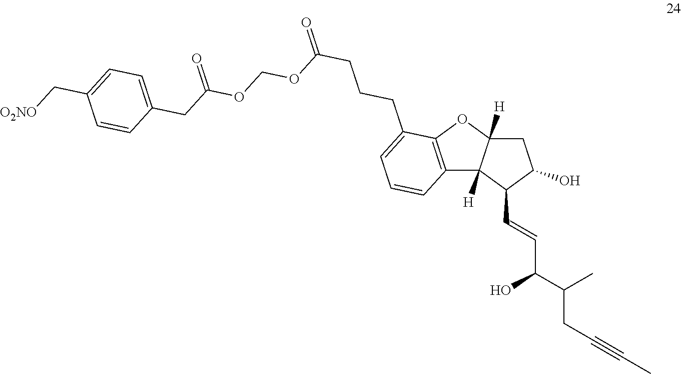

Referring to the synthesis method of Example 6, the compound 24 can be produced. ¹H NMR (300 MHz, DMSO-d) δ 7.54-7.38 (m, 2H), 7.30-7.15 (m, 2H), 7.15-7.02 (m, 2H), 6.97 (ddt, J=7.0, 2.0, 1.0 Hz, 1H), 5.97-5.70 (m, 4H), 5.50-5.26 (m, 2H), 4.94 (dt, J=5.1, 4.2 Hz, 1H), 4.80 (d, J=6.2 Hz, 1H), 4.26-4.04 (m, 3H), 3.62 (q, J=0.8 Hz, 2H), 2.77 (dqd, J=31.3, 6.5, 6.1, 1.1 Hz, 3H), 2.43 (td, J=7.1, 1.0 Hz, 2H), 2.27-1.82 (m, 7H), 1.60 (t, J=2.0 Hz, 3H), 1.04 (d, J=6.6 Hz, 3H).

### Example 25

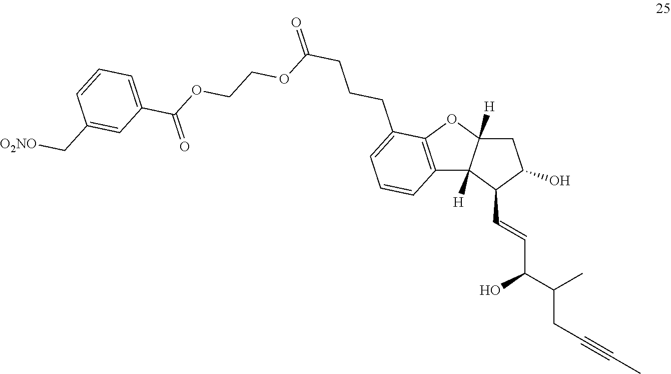

Referring to the synthesis method of Example 6, the compound 25 can be produced. ¹H NMR (300 MHz, DMSO-d) δ 8.04 (tt, J=2.2, 1.0 Hz, 1H), 7.92 (ddd, J=7.7, 2.2, 1.1 Hz, 1H), 7.57 (ddq, J=7.9, 2.2, 1.1 Hz, 1H), 7.47 (t, J=7.8 Hz, 1H), 7.20-7.02 (m, 2H), 6.97 (ddt, J=7.0, 2.0, 1.0 Hz, 1H), 5.89-5.70 (m, 2H), 5.45 (t, J=1.0 Hz, 2H), 4.94 (dt, J=5.0, 4.2 Hz, 1H), 4.80 (d, J=6.2 Hz, 1H), 4.46 (td, J=6.0, 0.8 Hz, 2H), 4.37 (td, J=6.0, 0.8 Hz, 2H), 4.26-4.04 (m, 3H), 3.42 (dd, J=5.5, 4.2 Hz, 1H), 2.93-2.62 (m, 3H), 2.41 (t, J=7.0 Hz, 2H), 2.21 (dp, J=5.9, 2.0 Hz, 2H), 2.16-1.84 (m, 5H), 1.65 (t, J=2.0 Hz, 3H), 1.02 (d, J=6.6 Hz, 3H).

### Example 26

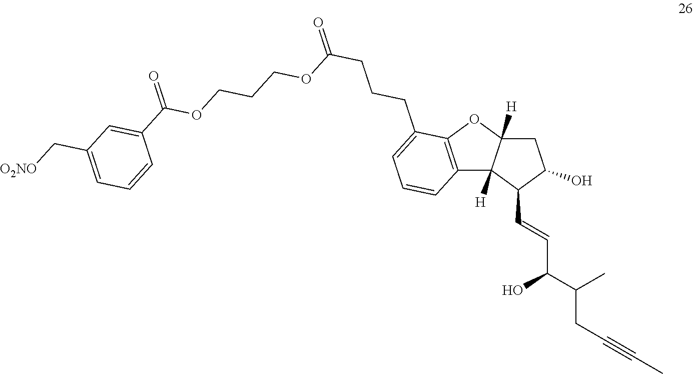

Referring to the synthesis method of Example 6, the compound 26 can be produced. ¹H NMR (300 MHz, DMSO-d) δ 8.11-7.84 (m, 2H), 7.65-7.37 (m, 2H), 7.13-7.01 (m, 2H), 6.97 (ddt, J=7.0, 2.0, 1.0 Hz, 1H), 5.92-5.62 (m, 2H), 4.94 (dt, J=5.0, 4.2 Hz, 1H), 4.80 (d, J=6.2 Hz, 1H), 4.32-3.95 (m, 7H), 3.45 (dd, J=5.6, 4.3 Hz, 1H), 2.89-2.67 (m, 3H), 2.40 (t, J=7.1 Hz, 2H), 2.21 (dp, J=5.9, 2.0 Hz, 2H), 2.18-1.85 (m, 7H), 1.61 (t, J=2.0 Hz, 3H), 1.00 (d, J=6.8 Hz, 3H).

### Example 27

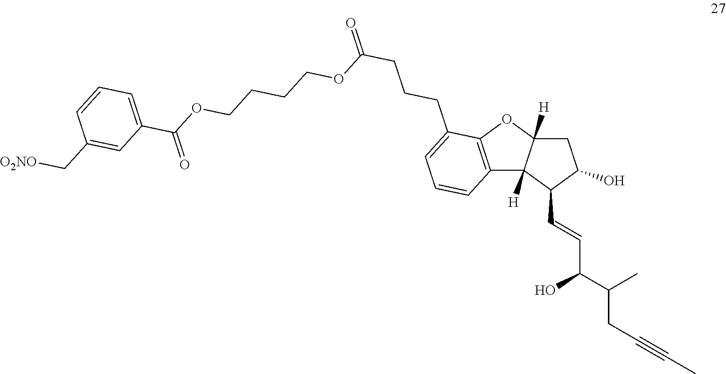

Referring to the synthesis method according to Example 6, the compound 27 can be produced. ¹H NMR (300 MHz, DMSO-d) δ 8.05-7.89 (m, 2H), 7.58-7.35 (m, 2H), 7.17-7.04 (m, 2H), 6.97 (ddt, J=6.9, 2.0, 1.0 Hz, 1H), 5.90-5.65 (m, 2H), 4.94 (dt, J=5.1, 4.2 Hz, 1H), 4.80 (d, J=6.2 Hz, 1H), 4.30-4.02 (m, 7H), 3.45 (dd, J=5.6, 4.3 Hz, 1H), 2.88-2.66 (m, 3H), 2.49-2.31 (m, 2H), 2.21 (dp, J=5.9, 2.0 Hz, 2H), 2.18-1.73 (m, 9H), 1.61 (t, J=2.2 Hz, 3H), 1.01 (d, J=6.4 Hz, 3H).

### Example 28

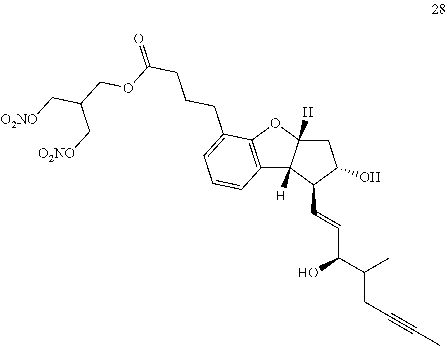

Referring to the synthesis method of Example 6, the compound 28 can be produced. ¹H NMR (300 MHz, DMSO-d) δ 7.22-7.06 (m, 2H), 7.00 (dtt, J=5.7, 3.5, 1.0 Hz, 1H), 5.78 (qd, J=15.6, 6.2 Hz, 2H), 4.94 (dt, J=5.1, 4.3 Hz, 1H), 4.62-4.40 (m, 4H), 4.29-4.05 (m, 4H), 3.43 (dd, J=5.5, 4.2 Hz, 1H), 2.87-2.67 (m, 3H), 2.55-2.39 (m, 3H), 2.26-2.16 (m, 2H), 2.16-1.85 (m, 5H), 1.66 (t, J=2.1 Hz, 3H), 1.00 (d, J=6.5 Hz, 3H).

### Example 29

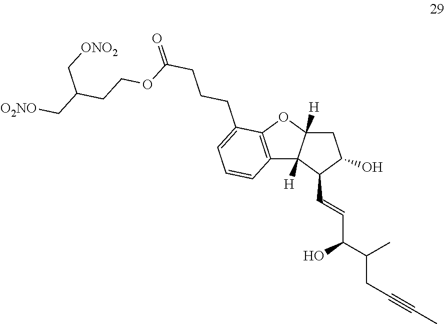

Referring to the synthesis method of Example 1, the compound 29 can be produced. ¹H NMR (300 MHz, DMSO-d) δ 7.19-7.07 (m, 2H), 7.01 (ddt, J=6.9, 1.9, 1.0 Hz, 1H), 5.78 (qd, J=15.6, 6.2 Hz, 2H), 4.94 (dt, J=5.0, 4.2 Hz, 1H), 4.56-4.36 (m, 4H), 4.29-4.01 (m, 4H), 3.92 (d, J=5.5 Hz, 1H), 2.91-2.65 (m, 3H), 2.40 (t, J=7.0 Hz, 2H), 2.33-2.17 (m, 3H), 2.17-1.81 (m, 7H), 1.60 (t, J=1.9 Hz, 3H), 0.99 (d, J=6.2 Hz, 3H).

### Example 30

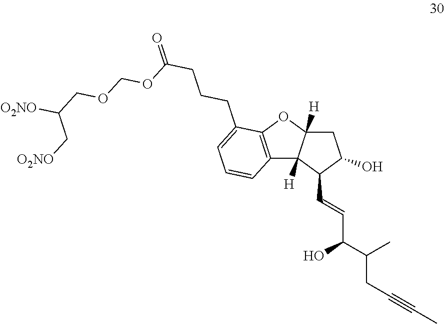

Referring to the synthesis method of Example 6, the compound 30 can be produced. ¹H NMR (300 MHz, DMSO-d) δ 7.21-7.07 (m, 2H), 7.00 (ddt, J=6.9, 1.9, 1.0 Hz, 1H), 5.78 (qd, J=15.6, 6.2 Hz, 2H), 5.28-5.02 (m, 3H), 4.94 (dt, J=5.1, 4.2 Hz, 1H), 4.74 (dd, J=21.4, 5.9 Hz, 3H), 4.23-4.05 (m, 2H), 3.93 (dd, J=5.5, 3.9 Hz, 3H), 3.42 (dd, J=5.5, 4.2 Hz, 1H), 2.87-2.67 (m, 3H), 2.43 (t, J=7.1 Hz, 2H), 2.21 (dp, J=6.2, 2.0 Hz, 2H), 2.18-1.84 (m, 5H), 1.60 (t, J=2.2 Hz, 3H), 1.00 (d, J=6.6 Hz, 3H).

### Example 31

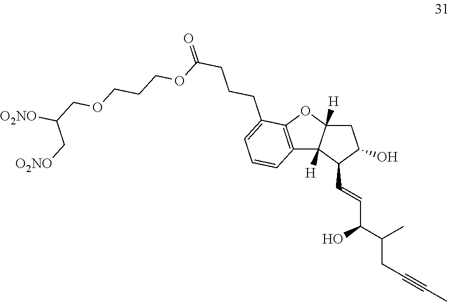

Referring to the synthesis method of Example 6, the compound 31 can be produced. ¹H NMR (300 MHz, DMSO-d) δ 7.15-7.04 (m, 2H), 6.97 (ddt, J=6.9, 1.9, 1.0 Hz, 1H), 5.78 (qd, J=15.6, 6.2 Hz, 2H), 5.11 (p, J=5.6 Hz, 1H), 4.94 (dt, J=5.0, 4.2 Hz, 1H), 4.68 (d, J=5.6 Hz, 2H), 4.27-4.02 (m, 5H), 3.84 (d, J=5.5 Hz, 2H), 3.56-3.29 (m, 3H), 2.86-2.63 (m, 3H), 2.40 (t, J=7.1 Hz, 2H), 2.21 (dp, J=5.9, 2.0 Hz, 2H), 2.17-1.88 (m, 7H), 1.61 (t, J=2.2 Hz, 3H), 0.99 (d, J=6.6 Hz, 3H).

### Example 32

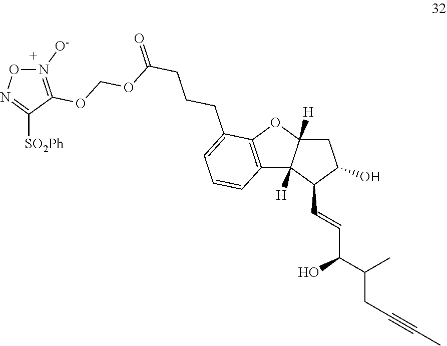

synthetic Method

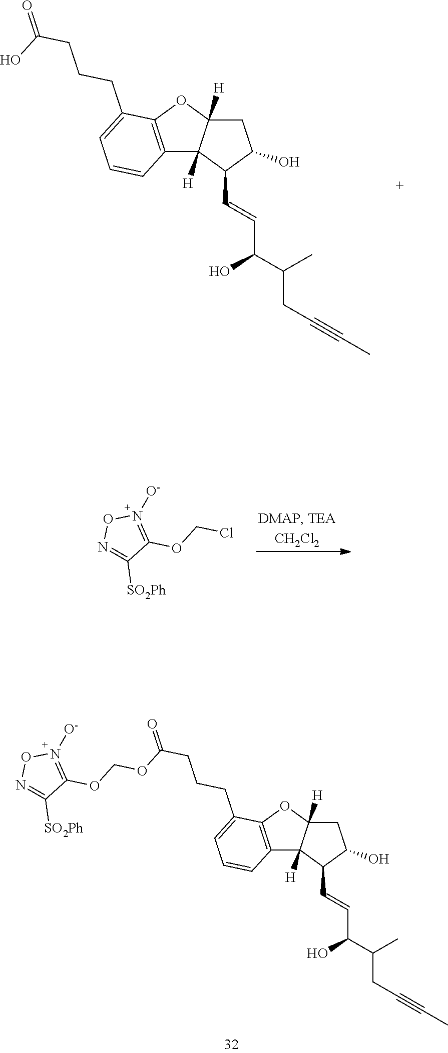

beraprost (60 mg), chloroformyl F za nitrogen oxide and D MAP, TEA were dissolved in 2 mL anhydrous dichloromethane, stirred at room temperature for four hours, diluted with 3 mL dichloromethane in the reaction solution, washed twice with 10% hydrochloric acid, washed once with saturated table salt, filtered, filtrate concentrated, and H PLC purified in Example 32.40% yield. ¹H NMR (300 MHz, DMSO-d) δ 7.94-7.73 (m, 2H), 7.69-7.50 (m, 3H), 7.13-7.02 (m, 2H), 6.97 (ddt, J=7.0, 2.0, 1.0 Hz, 1H), 6.11-5.95 (m, 2H), 5.89-5.64 (m, 2H), 4.94 (dt, J=5.0, 4.2 Hz, 1H), 4.80 (d, J=6.2 Hz, 1H), 4.22-4.03 (m, 3H), 3.45 (dd, J=5.6, 4.3 Hz, 1H), 2.88-2.65 (m, 3H), 2.43 (t, J=7.1 Hz, 2H), 2.21 (dp, J=5.9, 2.0 Hz, 2H), 2.18-1.84 (m, 5H), 1.61 (t, J=2.1 Hz, 3H), 1.00 (d, J=6.2 Hz, 3H).

### Example 33

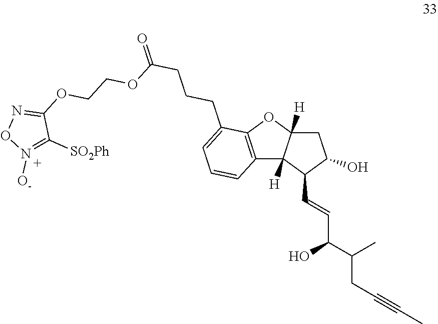

Referring to the synthesis method of Example 32, the compound 33 can be produced. ¹H NMR (300 MHz, DMSO-d) δ 8.04-7.90 (m, 2H), 7.73-7.49 (m, 3H), 7.17-7.02 (m, 2H), 6.97 (ddt, J=7.0, 2.0, 1.0 Hz, 1H), 5.93-5.61 (m, 2H), 4.94 (dt, J=5.0, 4.2 Hz, 1H), 4.63 (td, J=6.2, 1.0 Hz, 2H), 4.43 (t, J=6.2 Hz, 2H), 4.27-4.00 (m, 3H), 3.45 (dd, J=5.6, 4.3 Hz, 1H), 2.88-2.62 (m, 3H), 2.40 (t, J=7.1 Hz, 2H), 2.29-1.86 (m, 7H), 1.55 (t, J=2.1 Hz, 3H), 1.01 (d, J=6.2 Hz, 3H).

### Example 34

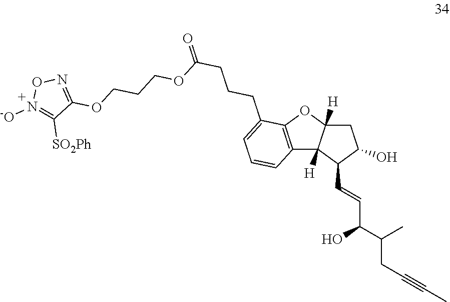

Referring to the synthesis method of Example 32, the compound 34 can be produced. ¹H NMR (300 MHz, DMSO-d) δ 8.12-7.91 (m, 2H), 7.70-7.54 (m, 3H), 7.17-7.04 (m, 2H), 6.97 (ddt, J=7.0, 2.0, 1.0 Hz, 1H), 5.89-5.63 (m, 2H), 4.94 (dt, J=5.0, 4.2 Hz, 1H), 4.80 (d, J=6.2 Hz, 1H), 4.43 (t, J=6.1 Hz, 2H), 4.24-4.03 (m, 5H), 3.45 (dd, J=5.6, 4.3 Hz, 1H), 2.88-2.67 (m, 3H), 2.40 (t, J=7.1 Hz, 2H), 2.27-1.85 (m, 9H), 1.57 (t, J=1.9 Hz, 3H), 0.99 (d, J=6.6 Hz, 3H).

### Example 35

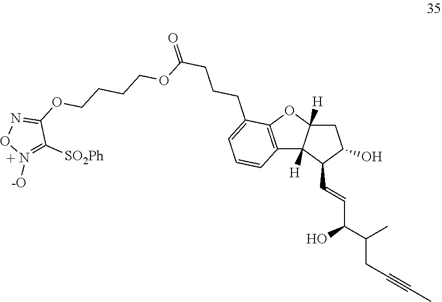

Referring to the synthesis method of Example 32, the compound 35 can be produced. ¹H NMR (300 MHz, DMSO-d) δ 8.04-7.84 (m, 2H), 7.70-7.52 (m, 3H), 7.16-7.04 (m, 2H), 6.97 (ddt, J=7.3, 1.8, 0.9 Hz, 1H), 5.90-5.63 (m, 2H), 4.94 (dt, J=5.1, 4.2 Hz, 1H), 4.47-4.31 (m, 2H), 4.24-4.00 (m, 5H), 3.45 (dd, J=5.6, 4.3 Hz, 1H), 2.89-2.62 (m, 3H), 2.48-2.34 (m, 2H), 2.21 (dp, J=5.9, 2.0 Hz, 2H), 2.20-1.87 (m, 5H), 1.87-1.72 (m, 4H), 1.62 (t, J=1.8 Hz, 3H), 0.99 (d, J=6.2 Hz, 3H).

### Test Example 1, the In Vitro NO Release Test of the Compound

NO was transiently oxidized in aqueous solution using released NO by Griess' method, NO²⁻ And Griess reagent form a complex, which has strong UV uptake at 540 nm to determine the amount of NO released by the compound.
1) Preparation of solution: blank solution: 10 mL DMSO and 190 m L PBS mixed; G riess Reagent: sulfonamide (4.0 g), N-(1-naphthalyl) ethylenediamine dihydrochloride (0.2 g) and 10 mL 85% H3PO4 dissolved in 90 mL of distilled water, Blow until clear solution; L-cysteine solution: after accurate weighing of L-cysteine, Add a certain amount of PBS, Prepare a weighed 200 μM solution; Test compound solution: the exact weighing of the test compound, Dissolved in D MSO and diluted to a concentration of 1 mM, Then, when diluted in PBS, To a concentration of 200 μM.

2) Formulation of standard curve equation: prepare sodium nitrite solution concentration: 0 ,0 , 1, 3, 6, 12.5, 25, 50, 100 μmol/L, 150 μL of each concentration, add 50 μL of Griess reagent mix, incubated in 37° C. constant temperature shaker for 30 min, the plate reader measured each tube absorbance at 540 nm, after subtracting the blank solution reading, the standard curve equation . . . 78.56.13.25
3) Test compound test: mix 2 mL of the test compound solution and L-cysteine solution, incubate in 37° C. constant temperature shakers for 120 min, take 150 μL of each mixture, add 50 μL of Griess reagent, then incubate in 37° C. constant temperature shaker for 30 min, the microplate measured each tube absorbance at 540 nm, then subtract the blank solution reading and substitute the value into the standard curve equation to obtain NO release.

By testing, the partial compound data of the invention are shown in Table 1, and the test results show that the nitric oxide donor beraprost derivatives or their medicinal salts in the invention have good NO release effect.

TABLE 1
Example NO release effects of the compound

| | 120 min NO²⁻ |
|---|---|
| Compounds | (μM) |
| 1 | 15.5 |
| 2 | 14.3 |
| 3 | 16.6 |
| 4 | 14.1 |
| 5 | 11.8 |
| 6 | 17.8 |
| 7 | 17.7 |
| 8 | 14.8 |
| 9 | 18.2 |
| 10 | 11.3 |
| 11 | 10.1 |
| 12 | 8.5 |
| 13 | 10.1 |
| 14 | 9.6 |
| 15 | 12.7 |
| 16 | 12.1 |
| 17 | 7.6 |
| 18 | 8.0 |
| 19 | 11.3 |
| 20 | 10.0 |
| 21 | 11.1 |
| 22 | 13.7 |
| 23 | 15.5 |
| 24 | 15.8 |
| 25 | 10.7 |
| 26 | 12.6 |
| 27 | 11.9 |
| 28 | 14.8 |
| 29 | 16.2 |
| 30 | 17.8 |
| 31 | 16.7 |
| 32 | 20.4 |
| 33 | 14.2 |
| 34 | 17.2 |
| 35 | 11.8 |

### Test Example 2, In Vivo Test in Rats with Hypoxic Pulmonary Hypertension

1) Experimental instruments and materials, HX-200 animal ventilator, and SD male rats used in the experiment were all purchased from Yangzhou University. All control groups were reared in a normal environment, and the administration intervention and model groups were reared in the low pressure low oxygen chamber (air pressure 50 kPa, oxygen concentration 10%).
2) Compound 15 was dissolved using DMSO/solutol/water (10/10/80). In the intervention group, the dose was 5 mg/kg, all rats were weighed weekly, survival was recorded, and pulmonary artery pressure was measured after four weeks. Rats (3 ml/kg) were anesthetized with chloral hydrate (100 g/L), fixed in the supine position, tracheotomy, and breathing assisted with a small animal ventilator (frequency 60 beats/min, 5 ml, breath ratio 4:5). Free the left third rib, a catheter connecting one end to the tension transducer was sent to the pulmonary artery, and the mean pulmonary artery pressure (mP AP) was recorded through the BL-420E Biofunction experimental system. The thoracic fluid and ascites were examined and collected, and finally the rats were killed by drawing blood from the abdominal aorta.
3) Experimental results: compared with the control group, mPAP in the model group was significantly increased, and mPAP in the intervention group administered compound 15 was lower compared with the model group, which had a good treatment effect of hypoxic pulmonary hypertension. The results of FIG. 1 show the effect of beraprost derivative compound 15.

### Test Example 3, the Therapeutic Effect of Osteoporosis

1) Test materials; 1. Animal clean grade C57BL/6 strain, 8-10 weeks old not pregnant females were purchased from Yangzhou University. 2. Main reagents and instruments, Bujiale (estradiol valerate tablets, Bayer); mouse osteocalcin (osteocalcin, OC) enzyme-linked immunotest kit, alkaline phosphatase (alkaline phosphatase, ALP) test kit, tartrate acid phosphatase (StrACP) test kit (all the above kits were purchased in Nanjing). Dual-energy X-ray bone densitometer (HOLOGIC); ECLIPSE 50i microscope (Nikon); MUTISKANMK3 microplate reader (Thermo); tissue slicing equipment (including KD-TS3D1 biological tissue automatic dehydrmachine (Zhejiang Cody), TB-718 biological tissue automatic embedding machine (Hubei), R138 rotary slicer (Hubei), TK-212 automatic constant temperature drift instrument (Hubei), TK-213 automatic constant temperature dryer (Hubei), etc.).
2) Test method: A animals were grouped, and the random method divided the female mice into 4 groups, namely, sham operation group, model group, positive drug group, and test drug group. B Preparation of postmenopausal osteoporosis mouse model, chloral hydrate anesthesia and removed the ovaries in the lying position. In the sham group, only the same volume of fat tissue near the ovaries was removed. Vaginal smear was performed on days 4-8 after ovariectomy to determine if the ovariectomy was complete. Method of c, on the third day after the operation, positive control drug (0.1 mL/10 g gavage), equal volume of 0.9%sodium chloride solution in sham group and model group, continuous gavage for 28 d. Test ResResults test: Blood and bone tissue samples after day 28. The following indicators were tested: (1) determination of bone density, lumbar L4-6 measurement of bone density by double energy X-ray bone density instrument. (2) Observation of bone tissue and morphology, the bone tissue and morphology changes were observed by tibial HE staining, mainly testing the trabecular volume ratio (bone volume/tissue volume, BV/TV) and the number of trabecular bone (trabecular bone number, Tb. N) and trabecular separation (trabecular separation, Tb. And sp) were the quantitative evaluation index. (3) For the determination of biological indicators ALP, StrACP, OC, and E 2 in mouse serum, blood was collected by enucleation method, and by kit and enzyme-linked adsorption assay. E All data were analyzed using the SPSS 20.0 software.
3) Test results: Compared with the model group, the test compound could effectively increase the amount of lumbar bone mineral density and osteocalcin in mice; the content of alkaline phosphatase and acid phosphatase in serum was significantly decreased, indicating that the compound could improve the relevant indicators in estrogen deficiency-induced osteoporosis.

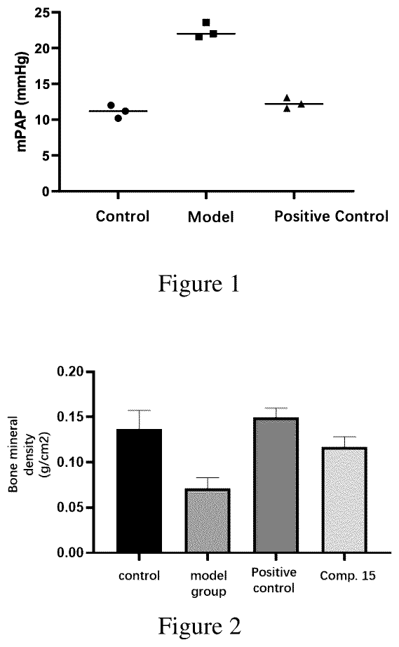

Among them, FIG. 2 shows bone density data in mice after treatment with nitric oxide beraprost derivative compound 15. Tables 2 and and and 33 show the data on quantitative indicators of bone morphology and bone metabolism indicators in serum of mice after treatment with compound 15.

TABLE 2
Quantitative index data of bone morphology

| | | Tb · N | Tb · sp |
|---|---|---|---|
| group | BV/VT | (mm²) | (μm) |
| The sham surgery group | 0.37 ± 0.06 | 8.2 ± 0.9 | 93 ± 12 |
| model set | 0.19 ± 0.04 | 4.9 ± 0.6 | 182 ± 15 |
| Positive drug group | 0.33 ± 0.04 | 8.8 ± 0.7 | 144 ± 19 |
| 200 μg/mL compound 15 | 0.29 ± 0.05 | 7.5 ± 0.6 | 152 ± 15 |

TABLE 3
Bone metabolic parameters in mouse serum

| | ALP | | | |
|---|---|---|---|---|
| | (King- | | | |
| | Armstrong | Str ACP | OC | E₂ |
| group | unit/100 mL) | (U/L) | (ng/mL) | (ng/mL) |
| The sham surgery group | 16 ± 1.5 | 39 ± 2.8 | 1.6 ± 0.2 | 44 ± 4 |
| model set | 27 ± 1.9 | 44 ± 3.2 | 4.0 ± 0.3 | 22 ± 3 |
| Positive drug group | 18 ± 2.1 | 38 ± 4.0 | 1.8 ± 0.3 | 40 ± 5 |
| 200 μg/mL compound 15 | 19 ± 2.2 | 41 ± 3.6 | 1.7 ± 0.2 | 35 ± 4 |

### Test Example 4, the Renal Tubular Protective Effect of Acute Renal Failure

1) Test materials, glycerol (MacLean Company); CCK-8 kit, Annexin V-PE cell apoptosis detection kit, superoxide dismutase activity detection kit, malondialdehyde detection kit (Biyun Biotech); first anti-cysteine aspartate protease, caspase 3 and 9, B lymphocytoma-2 gene, Bcl-2-associated X protein (BAX, anti-rabbit), TGF-β 1, smad 3 (abcam, USA). Protein gel imager, microplate reader, and flow cytometer (Beckman Coulter, Type CytoFLEX). Clean-grade SD rats were purchased from Yangzhou University.
2) Test method, the model of acute renal failure of rat hind limb injection was used. After 24 h, the level of BUN and Cr increased in rat serum, vascular necrosis of renal interstitium, and inflammatory cell infiltration were regarded as successful model establishment. Some renal tissues from model animals were taken for in vitro culture, renal tubular epithelial cells were isolated and identified, and test drug co-culture (0-24 hours) was added. Changes of various biochemical indicators and apoptosis were determined. All data were used for statistical analysis.
3) Test results, OD after culture (3, 6, 12, 24 h)₄₅₀, MDA activity, BCL 2 protein level, SOD activity, apoptosis rate, increased caspase3, caspase9, BAX, TGF-b1, Smad3 protein levels, and test compounds caused OD after 6 h of cell culture compared with the model group₄₅₀Higher, and decreased the apoptosis rate, SOD activity, BCL 2, TGF-β 1, Smad3 protein levels (P<0.05), and increased MDA activity, caspase3, caspase9, BAX protein levels. Conclusion Test compounds can promote tubular epithelial cells proliferation, inhibit apoptosis, inhibit oxidative stress, and slow the damage of renal tubular epithelial cells by inhibiting the expression of proapoptotic proteins.

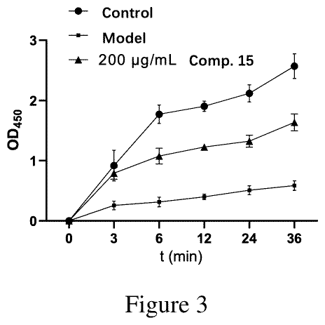

FIG. 3 shows the tubular proliferation of compound 15, Table 4 and Table 5 the effects of MDA, SOD and apoptosis rate, apoptotic protein and TGF-β 1 and Smad 3 in compound 15.

TABLE 4
Effect of compound 15 on MDA, SOD and apoptosis rate in
renal tubular epithelial cells in acute renal failure

| | | | The rate of |
|---|---|---|---|
| | MDA | SOD | apoptosis |
| group | (Nnol/g protein) | (U/g egg white) | (%) |
| control group | 511 ± 48 | 712 ± 65 | 7 ± 0.4 |
| model set | 262 ± 22 | 1432 ± 218 | 29 ± 3.2 |
| And 200 μg/mL of | 489 ± 38 | 1016 ± 117 | 9 ± 0.7 |
| compound in group 15 | | | |

TABLE 5
Effect of compound 15 on apoptotic protein and TGF- β 1
and Smad 3 in renal tubular epithelial cells in acute renal failure

| group | caspase3 | caspase9 | B CL2 | B AX | T GF-β1 | S mad3 |
|---|---|---|---|---|---|---|
| control group | 0.31 ± 0.08 | 0.39 ± 0.12 | 1.07 ± 0.13 | 0.29 ± 0.09 | 0.33 ± 0.02 | 0.11 ± 0.01 |
| model set | 1.27 ± 0.19 | 0.88 ± 0.2 | 0.08 ± 0.02 | 1.17 ± 0.17 | 0.91 ± 0.09 | 0.57 ± 0.08 |
| And 200 μg/mL of | 0.28 ± 0.06 | 0.48 ± 0.1 | 0.56 ± 0.11 | 0.32 ± 0.12 | 0.24 ± 0.05 | 0.19 ± 0.07 |
| compound in group 15 | | | | | | |

### Test Example 5, Antiplatelet Aggregation Effect

1) Experimental material: ADP, epinephrine, collagen (bilBio)
2) Experimental method: blood samples provided by healthy subjects were mixed blood with 3.8% citric acid solution and centrifuged at 160 r/min to obtain platelet-rich plasma. To calibrate the data, the obtained platelet-rich plasma was further centrifuged at 2000 r/min to obtain platelet-poor plasma, stored in a −20° C. refrigerator. Testing was performed using Born I's turbiometry. 225 μL of platelet rich plasma was added to the reaction cup, the embodiment compound was configured as 50 nM solution (25 m M Tris-acetate and 120 m M N a Cl), 25 μL of embodiment compound solution was added and after incubation at 37° C. for 2 min, 5 μL of ADP (final concentration 2 μM) was added for platelet aggregation induction. To assess the degree of platelet aggregation, the maximum absorbance value took the data after ADP, thus calculating the inhibition rate of ADP-induced platelet aggregation by each embodiment compound. According to the data in Table 6 below, the compounds of the present application embodiment all have a good effect of inhibiting the platelet aggregation induced by ADP.

TABLE 6
Example compound inhibits platelet aggregation induced by ADP

| | The rate of platelet |
|---|---|
| chemical | aggregation inhibition was |
| compound | observed (%) |
| 1 | 78 |
| 2 | 66 |
| 3 | 57 |
| 4 | 43 |
| 5 | 64 |
| 6 | 39 |
| 7 | 44 |
| 8 | 61 |
| 9 | 52 |
| 10 | 54 |
| 11 | 67 |
| 12 | 42 |
| 13 | 61 |
| 14 | 48 |
| 15 | 92 |
| 16 | 81 |
| 17 | 59 |
| 18 | 66 |
| 19 | 69 |
| 20 | 45 |
| 21 | 81 |
| 22 | 52 |
| 23 | 56 |
| 24 | 47 |
| 25 | 35 |
| 26 | 22 |
| 27 | 35 |
| 28 | 47 |
| 29 | 56 |
| 30 | 52 |
| 31 | 67 |
| 32 | 55 |
| 33 | 82 |
| 34 | 35 |
| 35 | 44 |

For those skilled in the art, the present disclosure is not limited to the aforementioned illustrative embodiments, but can be reflected in other specific forms without departing from its necessary attributes. It is therefore expected that all aspects are considered as illustrative rather than restrictive, embodiments with reference to the attached claims, not the aforementioned embodiment, references only to additional claims rather than the above examples, and all changes that fall within the meaning and scope of the equivalence of the claim are therefore expected to be included herein.

All patents, patent applications and references listed in this specification are referred here in their full context. In cases of inconsistency, including the definition of this disclosure would be persuasive.

1. A nitric oxide donor beraprost derivative or its medicinal salts as shown in formula I as follows: 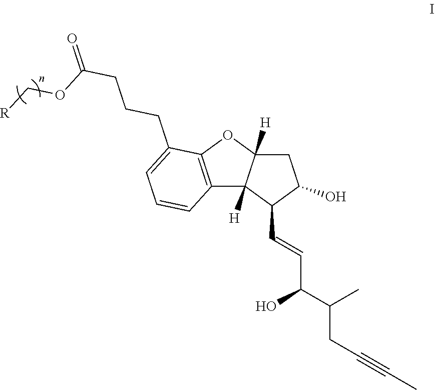
n is 0, 1 , 2, 3 or 4;
R is the —X—ONO₂, —OC(O)—X—ONO₂, —O—X—ONO₂, or 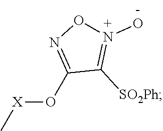
where X is a straight or branched chain of C₁-C₁₀ alkyl, cycloalkyl, or —C₁-C₁₀ alkyl-aromatic ring-; where C₁-C₁₀ alkyl, C₅₋₇ cycloalkyl or aryl ring may be substituted by one or more of the following substituents: halogen, hydroxyl, carboxyl, cyanogen, or —(C₁-C₁₀ alkyl)-ONO₂.

2. The nitric oxide donor beprost derivative or its medicinal salts according to claim 1, wherein the cyclic alkyl group is C₅₋₇ Cycloalkyl, and the aromatic ring is the C₅₋₁₀ aromatic ring.

3. The nitric oxide donor beprost derivative or medicinal salts thereof according to claim 1, comprising any of the following structures:

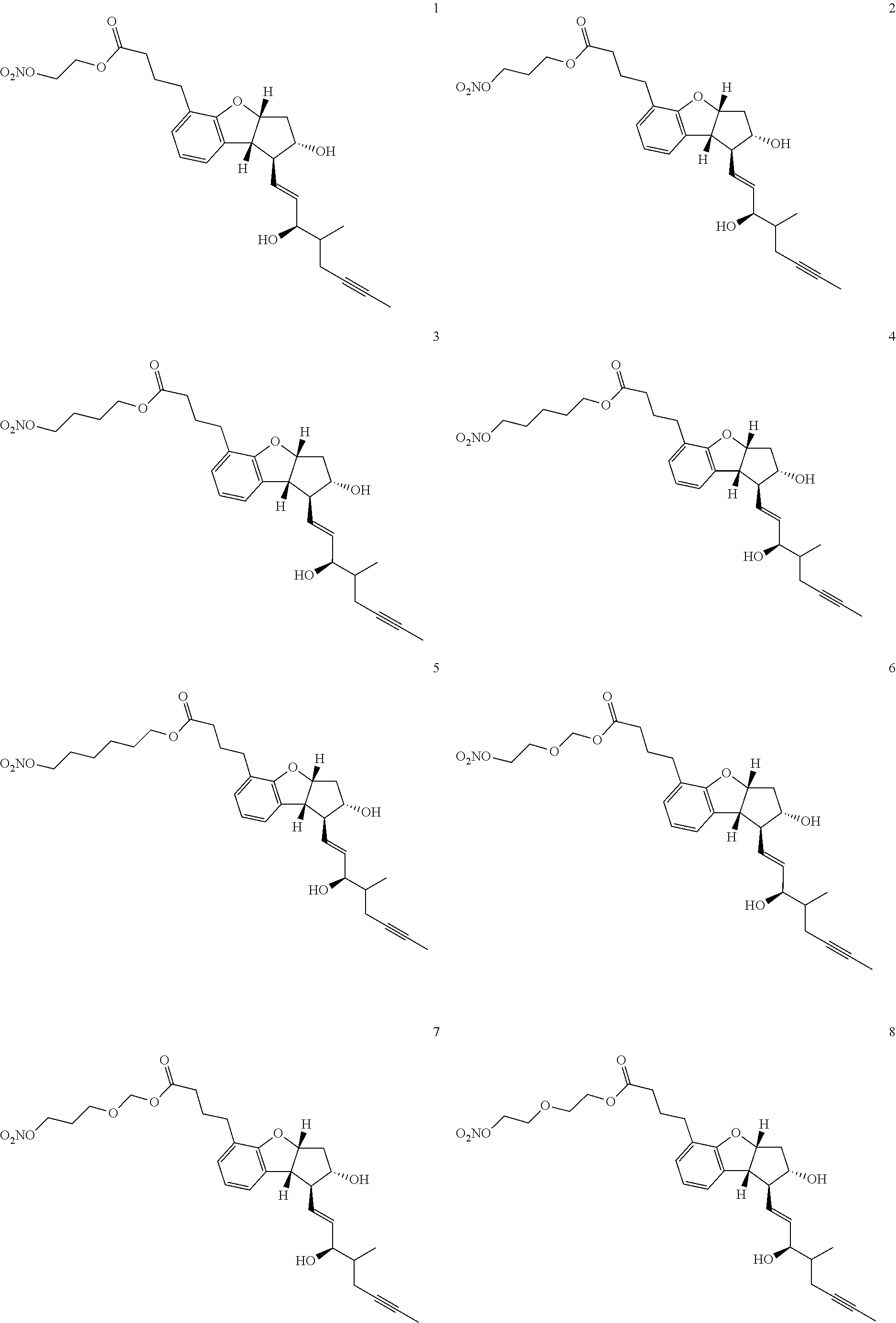

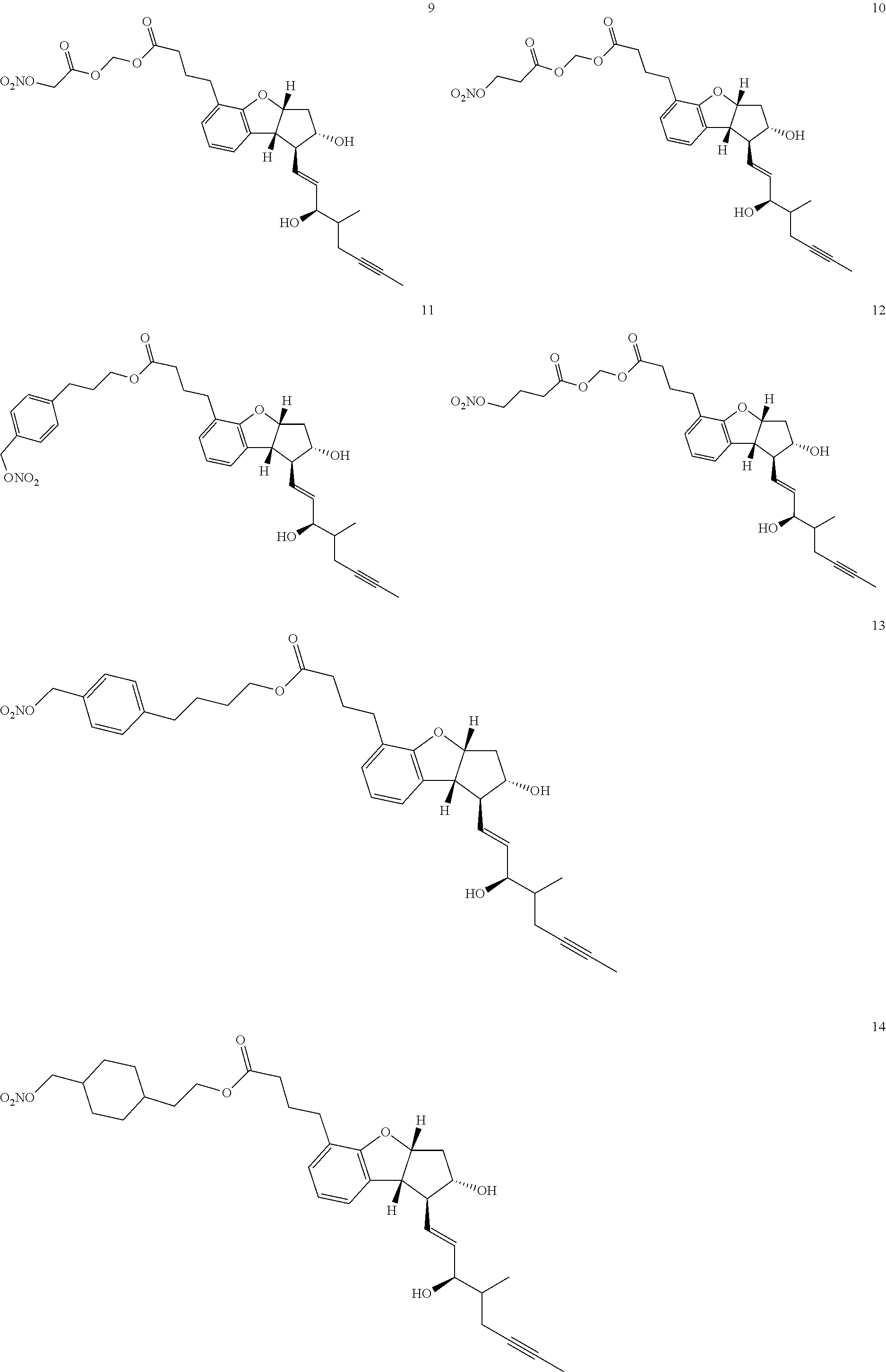

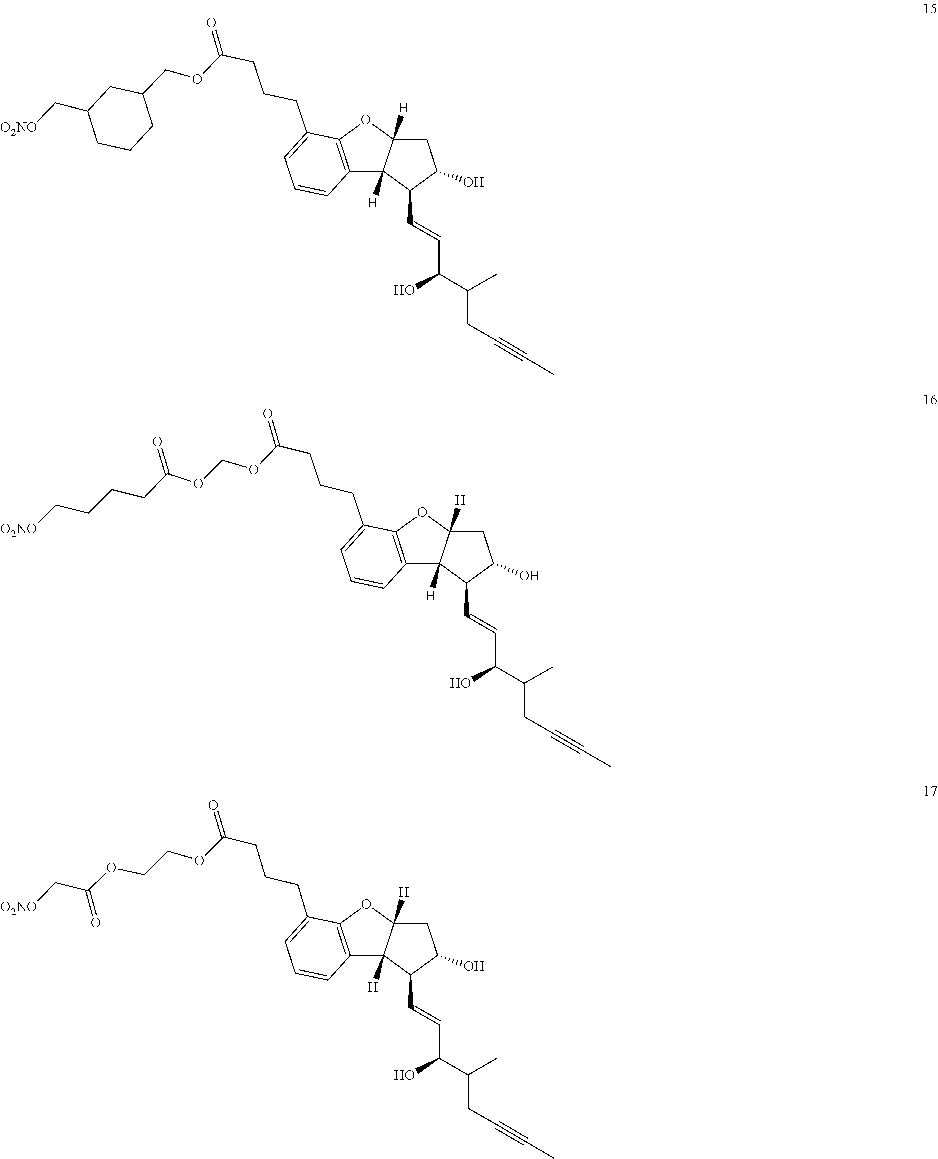

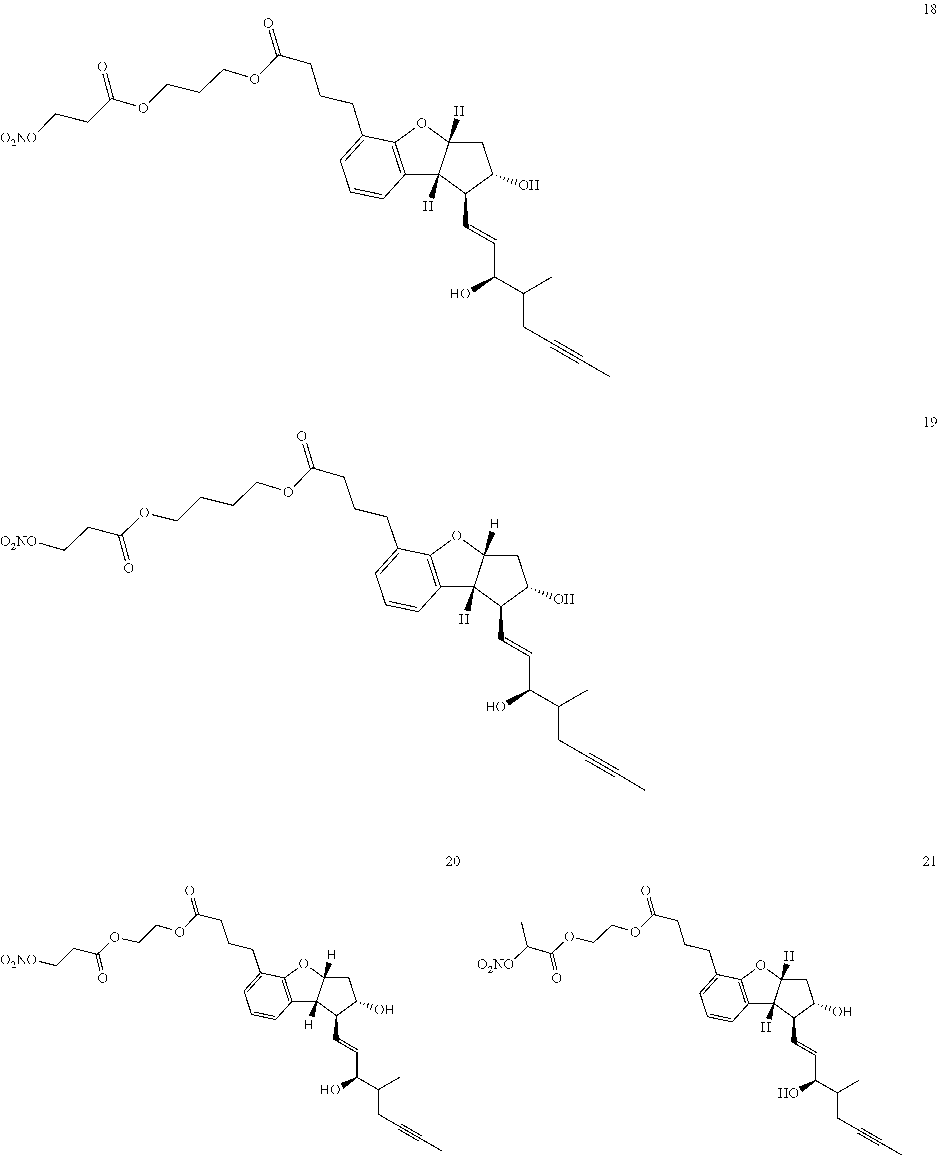

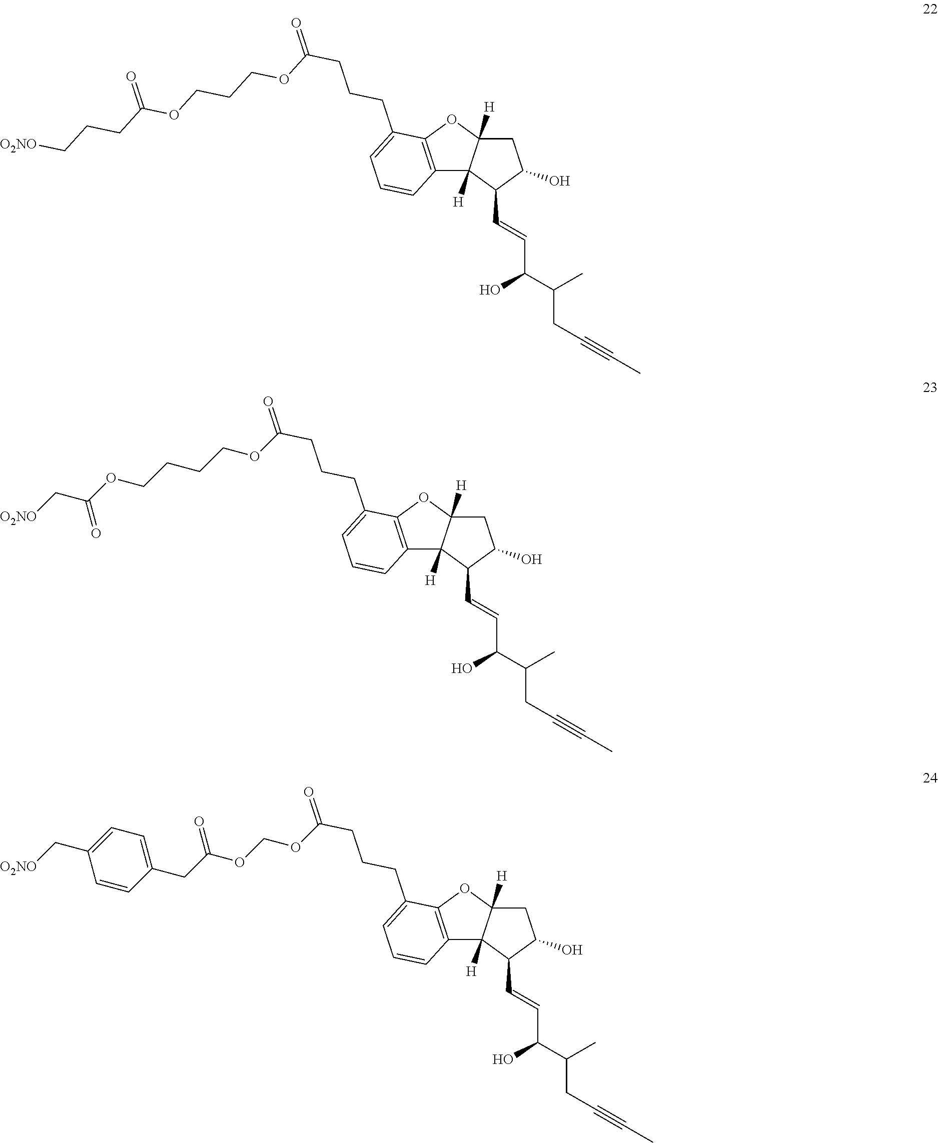

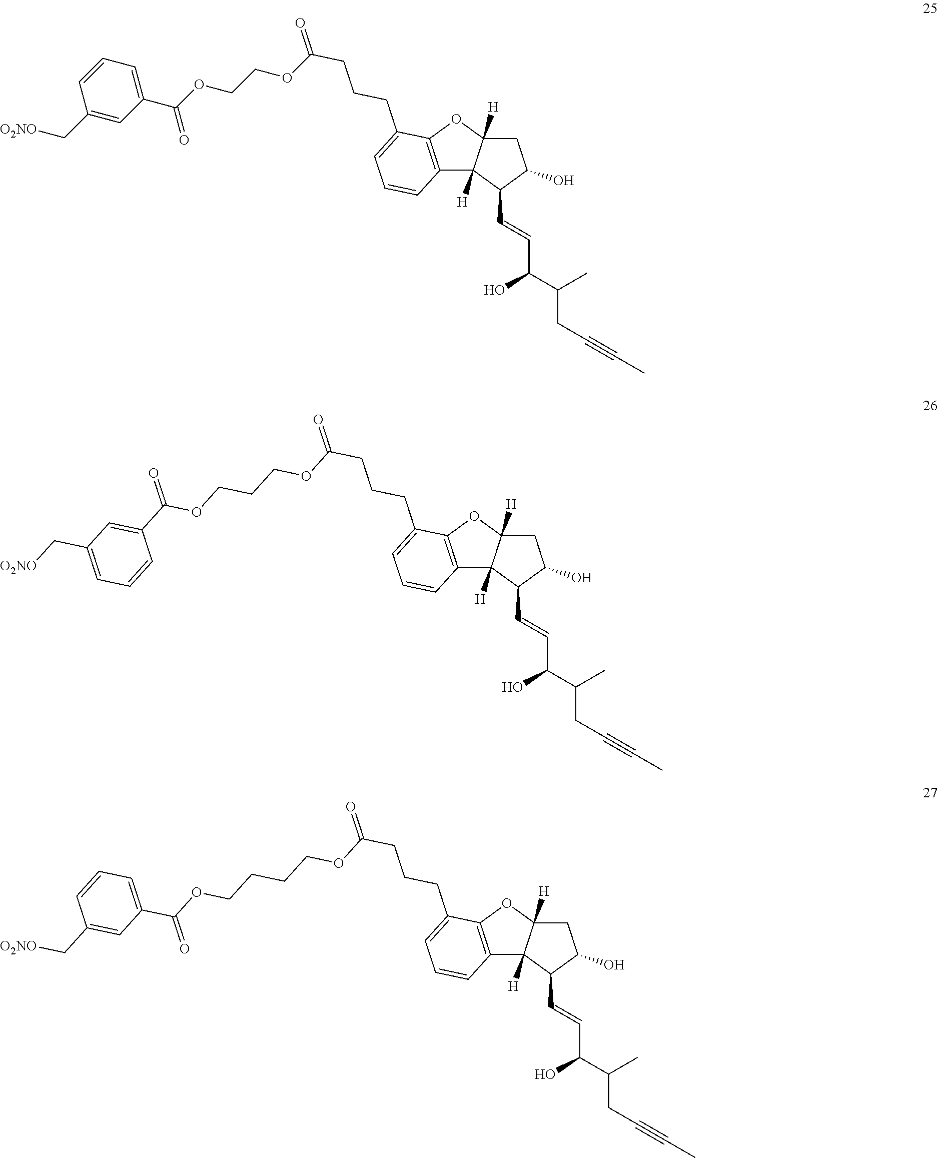

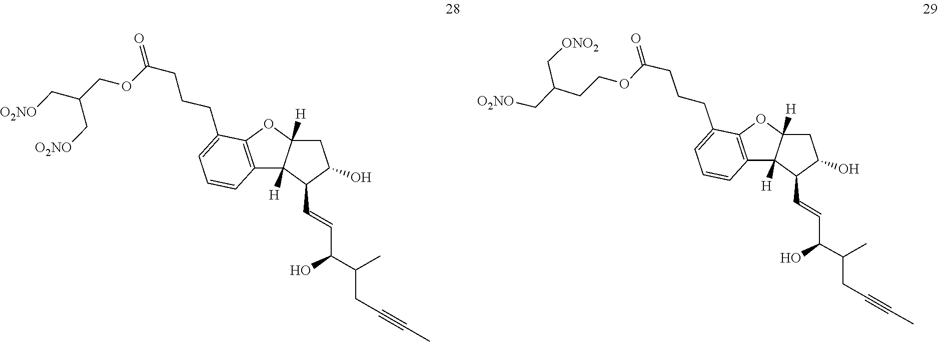

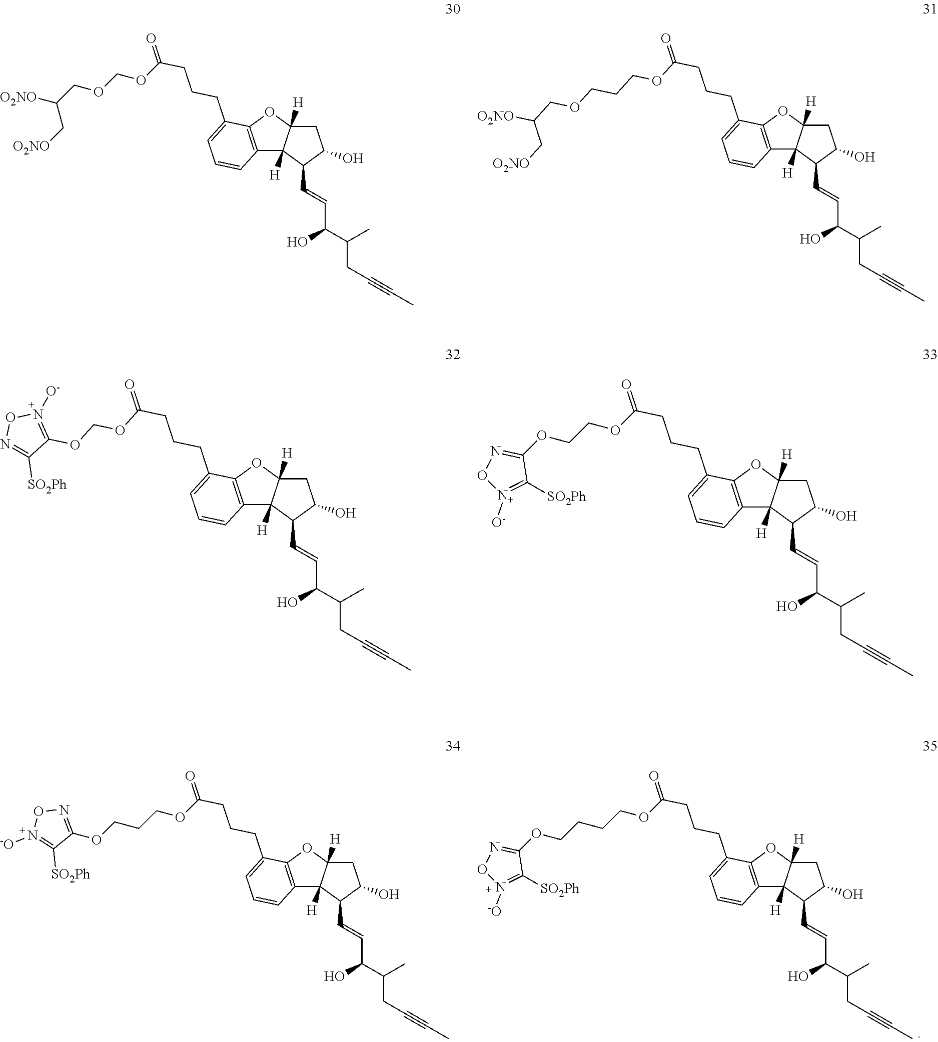

4. An application of a nitric oxide donor beraprost derivative or a medicinal salt thereof, as a procycoprinin analogue.

5. The application of the nitric oxide donor beraprost derivative or its medicinal salts of claim 1 in the preparation of various therapeutic drugs for the treatment of peripheral vascular diseases, including pulmonary hypertension, myocardial infarction, renal disease, occlusive arteriosclerosis, and ophthalmic diseases (such as diabetic fundus lesions, glaucoma, etc), osteoporosis, thrombotic angiitis, thromboembolic diseases.

6. A pharmaceutical composition comprising the nitric oxide donor beraprost derivative of claim 1 or a carrier acceptable for medicinal salts and drugs thereof.

7. The pharmaceutical composition according to claim 6, wherein the carrier is a mixture of one or more of a sustained-release agent, an excipient, agent, a filler, an adhesive, a wetting agent, a disintegration agent, an absorption promoter, an adsorption carrier, a surfactant, and a lubricant.

8. The pharmaceutical composition according to claim 6, wherein the pharmaceutical composition is either of the topical, oral, and injectable preparations.

9. A pharmaceutical composition according to claim 8, wherein the oral formulation is any of the granules, capsules, and tablets.
